(19) United States
(12) Patent Application Publication
(10) Pub. No.: US 2023/0338300 A1
(43) Pub. Date: Oct. 26, 2023

(54) PHARMACEUTICAL COMPOSITION CONTAINING COCRYSTALS FOR ADDITIVE MANUFACTURING

(71) Applicant: BOARD OF REGENTS, THE UNIVERSITY OF TEXAS SYSTEM, Austin, TX (US)

(72) Inventors: Mohammed MANIRUZZAMAN, Pflugerville, TX (US); Jiaxiang ZHANG, Pflugerville, TX (US); Rishi THAKKAR, Austin, TX (US)

(21) Appl. No.: 18/041,479

(22) PCT Filed: Aug. 14, 2021
(86) PCT No.: PCT/US21/46047

§ 371 Date: Feb. 13, 2023

Related U.S. Application Data
(60) Provisional application No. 63/065,829, filed on Aug. 14, 2020.

(51) Int. Cl. A61K 9/70; A61K 47/54; A61K 9/16; B33Y 70/00

(52) U.S. Cl. CPC A61K 9/70; A61K 47/542; A61K 47/545; A61K 9/1694; B33Y 70/00; B33Y 10/00

(57) ABSTRACT

The present disclosure provides methods of preparing pharmaceutical compositions using an additive manufacturing technique containing cocrystals. The cocrystals used in these compositions may be formed using an active pharmaceutical ingredient and a co-former. The co-former may be either an excipient or a second active pharmaceutical ingredient. These pharmaceutical compositions may be used in the treatment of a disease or disorder.

This application claims the benefit of priority to U.S. Provisional Application No. 63/065,829, filed on Aug. 14, 2020, the entire contents of which are hereby incorporated by reference.

### BACKGROUND

### 1. Field

The present disclosure relates generally to the field of pharmaceuticals and pharmaceutical manufacture. More particularly, it concerns compositions and methods of preparing a pharmaceutical composition comprising a cocrystal using additive manufacturing techniques.

### 2. Description of Related Art

Small molecular drug entities are the preferred choice for the treatment due to their low-cost of synthesis and ease of formulation as compared to large biomolecules. Although small molecules have been the major form of therapy, based on the pharmaceutical reports, around 40% APIs in the pipeline have very low water solubility which results in low bioavailability. Synthesis of cocrystals using solid-state processing such as hot-melt extrusion (HME) technologies is one optimal approach to improve the solubility of poorly soluble API with ease of scale-up and regulatory considerations. Cocrystals refer to solids that are crystalline single-phase materials composed of two or more different molecular crystalline compounds generally in a stoichiometric ratio which are neither solvates nor simple salts. One of the important advantages of co-crystals is that they generate a diverse array of solid-state forms for APIs that lack ionizable functional groups, which is a prerequisite for salt formation. The single phased cocrystals consist of physicochemical characters with conventional crystalline structures, that are: uniformity, the same macroscopic properties throughout the crystal; anisotropy, the crystals show different properties in different orientations; can form a polyhedron shape spontaneously; have a definite and obvious melting point; have a specific symmetry; and show diffraction effects to the X-ray and electron beam.

The process of manufacturing pharmaceutical cocrystals using HME that is through simultaneous melting and mixing of the target molecule and co-former via the use of a heated screw extruder technically is a continuous solid-state processing. The starting materials are fed in a definite molar ratio, and the melting will facilitate intimate mixing of the starting materials. The cocrystal nucleates directly in the melt, and pure cocrystal extrudate is isolated from the extruder continuously. These cocrystals can be used as an intermediate product instead of being regarded as a new API with or without downstream processing for any drug products manufacturing platforms, including additive manufacturing methods such as powder-based, filaments based or semi-solid based extrusion-based 3D printing.

Personalized drug development has gained significant interest in both the pharmaceutical industry and multiple research groups across the world, which has resulted in increased volumes of research publications and patents in this field. Additive manufacturing (AM), popularly recognized as 3D printing, is the formalized term for rapid prototyping (RP) that uses additive technologies and combines materials layer by layer from a computer-aided design (CAD) model. Unlike the traditional dosage manufacturing process, 3D printing can manufacture personalized products according to the inputs from patients, caregivers and professionals and products are made for immediate consumption. Nowadays, the development of pharmaceutical 3D printing is challenged by the limited materials available for such processes. For example, powder bed-based AM requires free-flowing powders whereas selective laser sintering (SLS) techniques require materials which are thermally stable and easy for sintering.

Therefore, there is an increasing need to develop methods that may be used to prepare pharmaceutical compositions using additive manufacturing techniques that contain cocrystals.

### SUMMARY

The present disclosure provides methods of preparing pharmaceutical compositions comprising one or more cocrystals using an additive manufacturing technique. In some aspects, the present disclosure provides methods of preparing a pharmaceutical composition comprising:
(A) obtain a composition comprising a co-crystal, wherein the co-crystal is of an active pharmaceutical ingredient and a co-former;
(B) subjecting the composition to an additive manufacturing technique to obtain a pharmaceutical composition.

In some embodiments, the composition comprises at least 50% of the active pharmaceutical ingredient and the co-former as a cocrystal. In some embodiments, the composition comprises at least 75% of the active pharmaceutical ingredient and the co-former as a cocrystal. In some embodiments, the composition comprises at least 90% of the active pharmaceutical ingredient and the co-former as a cocrystal. In some embodiments, the composition comprises at least 95% of the active pharmaceutical ingredient and the co-former as a cocrystal. In some embodiments, the composition comprises at least 97% of the active pharmaceutical ingredient and the co-former as a cocrystal. In some embodiments, the composition comprises at least 99% of the active pharmaceutical ingredient and the co-former as a cocrystal.

In some embodiments, the co-crystal comprises an active pharmaceutical ingredient and a co-former in a molar ratio from about 1:10 to about 10:1. In some embodiments, the molar ratio is from about 2:1 to about 1:2. In some embodiments, the molar ratio is about 2:1. In some embodiments, the molar ratio is about 1:1. In other embodiments, the molar ratio is about 1:2.

In some embodiments, the composition present as a filament, a powder, a granule, or a particle. In some embodiments, the composition is present as a filament. In other embodiments, the composition is present as a powder or a granule.

In some embodiments, the active pharmaceutical ingredient is a BCS Class II drug. In other embodiments, the active pharmaceutical ingredient is a BCS Class IV drug. In some embodiments, the active pharmaceutical ingredient is an active pharmaceutical ingredient with a melting point of less than 250° C. such as less than 200° C. In some embodiments, the active pharmaceutical ingredient is selected from anticancer agents, antifungal agents, psychiatric agents such as analgesics, consciousness level-altering agents such as anesthetic agents or hypnotics, nonsteroidal anti-inflammatory agents (NSAIDs), anthelmintics, antiacne agents, antianginal agents, antiarrhythmic agents, anti-asthma agents, antibacterial agents, anti-benign prostate hypertrophy agents, anticoagulants, antidepressants, antidiabetics, antiemetics, antiepileptics, antigout agents, antihypertensive agents, anti-inflammatory agents, antimalarials, antimigraine agents, antimuscarinic agents, antineoplastic agents, anti-obesity agents, antiosteoporosis agents, antiparkinsonian agents, antiproliferative agents, antiprotozoal agents, antithyroid agents, antitussive agent, anti-urinary incontinence agents, antiviral agents, anxiolytic agents, appetite suppressants, beta-blockers, cardiac inotropic agents, chemotherapeutic drugs, cognition enhancers, contraceptives, corticosteroids, Cox-2 inhibitors, diuretics, erectile dysfunction improvement agents, expectorants, gastrointestinal agents, histamine receptor antagonists, immunosuppressants, keratolytics, lipid regulating agents, leukotriene inhibitors, macrolides, muscle relaxants, neuroleptics, nutritional agents, opioid analgesics, protease inhibitors, and sedatives. In some embodiments, the active pharmaceutical ingredient is a chemotherapeutic drug, an antibiotic, or a nonsteroidal anti-inflammatory agent. In some embodiments, the active pharmaceutical ingredient is a chemotherapeutic drug. In other embodiments, the active pharmaceutical ingredient is an antibiotic. In other embodiments, the active pharmaceutical ingredient is a nonsteroidal anti-inflammatory agent.

In some embodiments, the co-former interacts with the active pharmaceutical ingredient through one or more non-covalent interactions. In some embodiments, the non-covalent interactions are ionic interactions, hydrogen bonding, halogen bonding, van der Waals forces, 7-7 interactions, or hydrophobic effects. In some embodiments, the co-former and the active pharmaceutical ingredient interact with two or more non-covalent interactions. In some embodiments, the co-former is a compound which modifies the solubility of the active pharmaceutical ingredient. In some embodiments, the co-former is a compound which is sparingly soluble and modifies the solubility of the active pharmaceutical ingredient. In some embodiments, the co-former is a compound which is sensitive to the environment and modifies the solubility of the active pharmaceutical ingredient. In some embodiments, the compound is sensitive to the pH of the environment. In other embodiments, the compound is sensitive to the temperature of the environment. In some embodiments, the co-former is a compound that has no therapeutic effect.

In other embodiments, the co-former is a second active pharmaceutical ingredient. In some embodiments, the second active pharmaceutical ingredient is for the same disease or disorder as the first active pharmaceutical ingredient. In other embodiments, the second active pharmaceutical ingredient is for a different disease or disorder as the first active pharmaceutical ingredient.

In some embodiments, the co-former comprises one or more functional groups selected from amine, amide, a nitrogen containing heterocycle, carbonyl, carboxyl, hydroxyl, phenol, sulfone, sulfine, sulfinyl, sulfonyl, mercapto, and methyl thio. In some embodiments, the functional group is a NH₂, OH, C(O), C(O)OH, SH, or a nitrogen containing heterocycle. In some embodiments, the functional group is a nitrogen containing heterocycle, NH₂, OH, or SH.

In some embodiments, the co-former is a flavoring compound such assaccharin. In other embodiments, the co-former is a carboxylic acid such as maleic acid. In other embodiments, the co-former is a vitamin or a vitamin derivative such as nicotinamide. In some embodiments, the pKₐ of the active pharmaceutical ingredient and the pKₐ of the co-former have a pKₐ difference of less than 3. In some embodiments, the pKₐ difference is less than 2. In some embodiments, the pKₐ difference is less than 1. In some embodiments, the pKₐ difference is less than 0.5.

In some embodiments, the composition further comprises an excipient. In some embodiments, the excipient is a pharmaceutically acceptable thermoplastic polymer. In some embodiments, the active pharmaceutical ingredient or the co-former is not soluble in the pharmaceutically acceptable thermoplastic polymer. In some embodiments, the active pharmaceutical ingredient and the co-former are not soluble in the pharmaceutically acceptable thermoplastic polymer. In some embodiments, the co-crystal has been prepared using hot-melt extrusion. In other embodiments, the co-crystal has been prepared using a solvent evaporation method.

In some embodiments, the additive manufacturing technique is vat photopolymerization, material jetting, binder jetting, powder-bed fusion, material extrusion, directed energy deposition, or sheet lamination. In some embodiments, the additive manufacturing technique is fused deposition modeling, binder spraying, or selective laser sintering.

In some embodiments, the additive manufacturing technique comprises exposing the composition to an energy source to form a pattern. In some embodiments, the pattern is prepared by passing the energy source over the composition with a print speed from about 0.1 mm/s to about 50,000 mm/s. In some embodiments, the print speed is from about 0.5 mm/s to about 1,000 mm/s. In some embodiments, the print speed is from about 1 mm/s to about 250 mm/s. In some embodiments, the print speed is 1 mm/s, 10 mm/s, 25 mm/s, 50 mm/s, or 75 mm/s.

In some embodiments, the energy source has a hatch spacing from about 0.1 μm to about 250 μm. In some embodiments, the hatch spacing is from about 10 μm to about 200 μm. In some embodiments, the hatch spacing is from about 10 μm to about 150 μm. In some embodiments, the hatch spacing is about 25 μm or 120 μm.

In some embodiments, the methods comprise exposing the composition to a laser in a pattern. In some embodiments, the methods comprise depositing a layer of the composition onto a surface in a chamber. In some embodiments, the layer has a layer thickness from about 1 μm to about 5 mm. In some embodiments, the layer thickness is from about 10 μm to about 2.5 mm. In some embodiments, the layer thickness is from about 50 μm to about 1 mm. In some embodiments, the layer thickness is from 50 μm to about 400 μm.

In some embodiments, the layer comprises a surface temperature at its surface different from a chamber temperature in the chamber. In some embodiments, the surface temperature is from about 0° C. to about 250° C. In some embodiments, the surface temperature is from about 50° C. to about 175° C. In some embodiments, the surface temperature is from about 75° C. to about 150° C. In some embodiments, the chamber temperature is from about 25° C. to about 250° C. In some embodiments, the chamber temperature is from about 50° C. to about 200° C. In some embodiments, the chamber temperature is from about 75° C. to about 150° C. In some embodiments, the surface temperature is more than 5° C. less than the melting point of the co-crystal. In some embodiments, the surface temperature is more than 10° C. less than the melting point of the composition.

In some embodiments, the energy source is a laser. In some embodiments, the laser comprises a laser power from about 0.1 W to about 250 W. In some embodiments, the laser power is from about 5 mW to about 20 W. In some embodiments, the laser power is from about 50 mW to about 1 W. In some embodiments, the laser power is from about 100 mW to about 500 mW. In some embodiments, the laser comprises a beam size from about 0.25 μm to about 1 mm. In some embodiments, the beam size is from about 1 μm to about 500 μm. In some embodiments, the beam size is from about 2.5 μm to about 100 μm. In some embodiments, the laser has a wavelength from about 50 nm to about 15,000 nm. In some embodiments, the wavelength is from about 5 nm to about 11,000 nm. In some embodiments, the wavelength is from about 200 nm to about 1,000 nm. In some embodiments, the laser gives the composition an amount of energy equal to an electron laser density from about 2.5 J/mm³ to about 500 J/mm³. In some embodiments, the electron laser density is from about 5 J/mm³ to about 250 J/mm³. In some embodiments, the electron laser density is from about 7.5 J/mm³ to about 50 J/mm³. In some embodiments, the electron laser density is greater than 2.5 J/mm³. In some embodiments, the electron laser density is greater than 5 J/mm³. In some embodiments, the electron laser density is greater than 7.5 J/mm³.

In some embodiments, the additive manufacturing technique comprises a method comprising:
(A) fusing the composition to obtain a fused composition; and
(B) extruding the fused composition through an extruder to obtain an extruded composition;
wherein the extruded composition is used to build an object and the object comprises a pattern.

In some embodiments, the object is built in a layer by layer fashion. In some embodiments, the extruder comprises a feeding step motor. In some embodiments, the feeding step motor comprises a feeding gear, a hot end, and a nozzle. In some embodiments, the pattern is prepared by passing the extruder over the composition. In some embodiments, the extruder is passed over the composition with a print speed from about 0.1 mm/s to about 50,000 mm/s. In some embodiments, the print speed is from about 0.5 mm/s to about 1,000 mm/s. In some embodiments, the print speed is from about 1 mm/s to about 250 mm/s. In some embodiments, the print speed is 1 mm/s, 10 mm/s, 25 mm/s, 50 mm/s, or 75 mm/s.

In some embodiments, the pattern is printed with a hatch speed from about 0.1 μm to about 1 mm. In some embodiments, the hatch spacing is from about 1 μm to about 500 μm. In some embodiments, the hatch spacing is from about 10 μm to about 250 μm. In some embodiments, the hatch spacing is about 25 μm or 120 μm. In some embodiments, the object is built upon a building platform. In some embodiments, the building platform is moved along in a Z-axis. In some embodiments, the building platform is heated to a first platform temperature from about 0° C. to about 250° C. In some embodiments, the first platform temperature is from about 50° C. to about 175° C. In some embodiments, the first platform temperature is from about 75° C. to about 150° C. In some embodiments, the building platform is heated to a second platform temperature from about −125° C. to about 25° C. In some embodiments, the second platform temperature is from about −100° C. to about 0° C. In some embodiments, the second platform temperature is from about −50° C. to about 0° C. In some embodiments, the second platform temperature is sufficient to cool or solidify the object.

In some embodiments, the extruded composition is a filament. In some embodiments, the methods comprise using the filament in a fusion deposition modeling to obtain the object. In some embodiments, the filament has a diameter from about 0.5 mm to about 10 mm. In some embodiments, the diameter is from about 1 mm to about 7.5 mm. In some embodiments, the diameter is from about 1.5 mm to about 5 mm. In some embodiments, the diameter is either 1.75 mm or 3 mm.

In some embodiments, the filament has a strength such that the force needed to break the filament is greater than 1000 g. In some embodiments, the strength of the filament is greater than 2000 g. In some embodiments, the strength of the filament is greater than 3000 g. In some embodiments, the filament has a strength such that the force needed to cut the filament is greater than 100 g. In some embodiments, the strength of the filament is greater than 200 g. In some embodiments, the strength of the filament is greater than 300 g. In some embodiments, the filament has a stress such that the force needed to beak the filament is greater than 5,000 g/mm². In some embodiments, the stress of the filament is greater than 10,000 g/mm². In some embodiments, the stress of the filament is greater than 15,000 g/mm². In some embodiments, the filament has a bend angle such that the force needed to break the filament is greater than 10°. In some embodiments, the strength of the filament is greater than 20°. In some embodiments, the strength of the filament is greater than 30°.

In some embodiments, the filament comprises an active pharmaceutical ingredient and an excipient. In some embodiments, the filament comprises from about 10% w/w to about to 99% w/w of the active pharmaceutical ingredient.

In some embodiments, the additive manufacturing technique comprises a method comprising:
(A) depositing the composition to form a powder; and
(B) depositing a liquid binding material onto the powder.

In some embodiments, the methods comprise repeating steps A and B. In some embodiments, steps A and B are repeated sufficient to create a pattern. In some embodiments, the pattern is prepared by passing a binder jetting head over the composition with a print speed from about 0.1 mm/s to about 50,000 mm/s. In some embodiments, the print speed is from about 0.5 mm/s to about 1,000 mm/s. In some embodiments, the print speed is from about 1 mm/s to about 250 mm/s. In some embodiments, the print speed is 1 mm/s, 10 mm/s, 25 mm/s, 50 mm/s, or 75 mm/s. In some embodiments, the binder jetting head has a hatch spacing from about 50 μm to about 1 mm. In some embodiments, the hatch spacing is from about 60 μm to about 500 μm. In some embodiments, the hatch spacing is from about 80 μm to about 400 μm. In some embodiments, the hatch spacing is about 100 μm.

In some embodiments, the liquid binding material has a viscosity from 0.1 mPa*s to 230 mPa*s. In some embodiments, the viscosity is from about 0.25 mPa*s to 150 mPa*s. In some embodiments, the viscosity is from about 0.5 mPa*s to 50 mPa*s. In some embodiments, the powder has a particle size D₅₀ of the powder is less than about 50 μm. In some embodiments, the powder has a particle size D₉₀ of the powder is greater than 50 μm. In some embodiments, particle size D₉₀ of the powder is less than 100 μm. In some embodiments, the particle size D₉₀ of the powder is less than 80 μm.

In some embodiments, the powder has a Carr's Index of less than 25%. In some embodiments, the Carr's Index is from about 15% to about 25%. In some embodiments, the angle of repose of the powder is less than 40°. In some embodiments, the angle of repose is from about 10° than 40°. In some embodiments, the angle of repose is from about 15° than 30°. In some embodiments, the angle of repose is from about 20° than 25°. In some embodiments, the powder has a bulk density below 5 g/cm³. In some embodiments, the bulk density is below 2.5 g/cm³. In some embodiments, the bulk density is below 1.5 g/cm³.

In some embodiments, the pharmaceutical composition further comprises a filler. In some embodiments, the filler is a salt such as a calcium salt. In some embodiments, the calcium salt is calcium sulfate. In some embodiments, the pharmaceutical composition comprises from about 1% w/w to about 99% w/w of filler. In some embodiments, the pharmaceutical composition comprises from about 25% w/w to about 98% w/w of filler. In some embodiments, the pharmaceutical composition comprises from about 50% w/w to about 98% w/w of filler. In some embodiments, the pharmaceutical composition comprises from about 75% w/w to about 97% w/w of filler.

In some embodiments, the pharmaceutical composition is deposited into a unit dose form. In some embodiments, the unit dose form comprises two or more of distinct domains. In some embodiments, each domain comprises a circular shape. In some embodiments, each domain comprises a height, a porosity, and either a core diameter for the central domain or an inner and outer diameter for domains around the core domain. In some embodiments, the height is from 0.1 mm to about 50 mm. In some embodiments, the height is from about 1 mm to about 25 mm. In some embodiments, the height is from about 2.5 mm to about 10 mm. In some embodiments, the porosity is from about 10% to about 100%. In some embodiments, the porosity is from about 20% to about 90%. In some embodiments, the porosity is from about 30% to about 80%. In other embodiments, the porosity is from about 60% to about 100%. In other embodiments, the porosity is from about 70% to about 100%.

In some embodiments, the core diameter is from about 0.1 mm to about 25 mm. In some embodiments, the core diameter is from about 0.5 mm to about 10 mm. In some embodiments, the core diameter is from about 1 mm to about 10 mm. In some embodiments, the core diameter is equal to the inner diameter of the second domain. In some embodiments, the inner diameter of the next domain is equal to the outer diameter of the preceding domain. In some embodiments, the inner diameter is from about 0.1 mm to about 50 mm. In some embodiments, the inner diameter is from about 0.5 mm to about 20 mm. In some embodiments, the inner diameter is from about 1 mm to about 20 mm. In some embodiments, the outer diameter is from about 0.2 mm to about 100 mm. In some embodiments, the outer diameter is from about 1 mm to about 40 mm. In some embodiments, the outer diameter is from about 2 mm to about 40 mm. In some embodiments, the unit dose form comprises 2, 3, 4, or 5 domains. In some embodiments, each domain has a different shape, porosity, height, or diameter.

In some embodiments, the pharmaceutical composition is a dosage form. In some embodiments, the methods further comprise milling the pharmaceutical composition into a dosage form. In some embodiments, the dosage form is formulated for oral, pulmonary, nasal, topical, transdermal, or parenteral delivery. In some embodiments, the dosage form is formulated for oral delivery such as a tablet, capsule, or suspension. In other embodiments, the dosage form is formulated for topical delivery such as an emulsion, ointment, or cream. In other embodiments, the dosage form is formulated for parenteral delivery such as a suspension, microemulsion, or depot.

In yet another aspect, the present disclosure provides pharmaceutical compositions prepared according to the methods described herein.

In still yet another aspect, the present disclosures provides methods of treating or preventing a disease or disorder in a patient comprising administering to the patient in need thereof a therapeutically effective amount of a composition prepared according to the methods described herein; wherein the active pharmaceutical ingredient is sufficient to treat or prevent the disease or disorder.

Other objects, features, and advantages of the present disclosure will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1 shows the process schematic demonstration of manufacturing the ibuprofen-saccharin cocrystal using the HME process.

FIG. 2 shows the PXRD of the co-crystal HME of saccharin and indomethacin.

FIGS. 3A-3C show the demonstration of the cocrystal loaded modulated medical devices (FIG. 3A) design of each component, (FIG. 3B) overview of the assembled medical devices, and (FIG. 3C) detailed view of each part of the medical device.

FIG. 4 shows the schematic picture of the manufacturing cocrystal loaded tablets using SLS.

FIG. 5 shows the DSC of SLS printed tablets depicting the presence of IND-SAC cocrystals (orange peak).

FIGS. 6A-6F shows the PLM and hot stage microscopy results of the individual species and the cocrystals FIGS. 6A&6D represent Indomethacin, FIGS. 6B&6E represent Saccharine and FIGS. 6C&6F represents IND-SAC cocrystals.

FIG. 7 shows the PXRD of IND-SAC 3D printed tablets.

FIG. 8 shows a schematic of a fused deposition modeling apparatus.

FIG. 9 shows a demonstration of the screw configuration and temperature profiles used herein.

FIG. 10 shows a demonstration of the 3D structure design of the tablets and the printing condition of the SLS process.

FIG. 11 shows a demonstration of the IBU, NTM, and IBU-NTM cocrystals molecules.

FIG. 12 shows the PLM figures of IBU, NTM, physical mixtures, and IBU-NTM cocrystals.

FIG. 13 shows the DSC curves of IBU, NTM, physical mixtures, and IBU-NTM cocrystals.

FIG. 14 shows the PXRD curves of IBU, NTM, physical mixtures, and IBU-NTM cocrystals.

FIG. 15 shows the FTIR curves of IBU, NTM, physical mixtures, and IBU-NTM cocrystals.

FIG. 16 shows the raman spectra of IBU, NTM, physical mixtures, and IBU-NTM cocrystals.

FIG. 17 shows a demonstration of the tablets prepared using different techniques and materials.

FIG. 18 shows a in vitro drug release profiles of printed tablets and directly compressed tablets.

FIGS. 19A-19C show (FIG. 19A) demonstration of the screw configuration and temperature profiles used in this work; (FIG. 19B) solid cocrystals of IBU-NTM were obtained when set zone 6-8 at room temperature; (FIG. 19C) molten materials obtained when zone 6-8 were set at a higher temperature.

FIG. 20 shows a top and front view of different infill patterns and infill densities of the tablets, and the different orientation under the texture analysis studies.

FIG. 21 shows the optical microscopy picture showed the blank and cocrystal-loaded tablets with different infill patterns and infill densities.

FIG. 22 shows a in vitro drug release profiles of cocrystal loaded tablets with different infill patterns and infill densities.

### DESCRIPTION OF ILLUSTRATIVE EMBODIMENTS

In some aspects, the present disclosure relates to the methods of preparing pharmaceutical compositions through an additive manufacturing technique containing a cocrystal of the active pharmaceutical ingredient and a conformer. The cocrystal made be prepared using a variety of different methods including solvent evaporation or hot melt extrusion. These co-crystals may then be utilized in the additive manufacturing processes. These types of pharmaceutical composition containing co-crystals have not been produced using these types of manufacturing methods. These and more details are described below.

### I. Pharmaceutical Compositions

In some aspects, the present disclosure provides methods of using additive manufacturing methods to prepare pharmaceutical compositions containing an active pharmaceutical ingredient or a pharmaceutically acceptable salt, ester, derivative, analog, pro-drug, or solvates thereof and a co-former which may be an excipient or a second active pharmaceutical ingredient as a co-crystal. These compositions may be used to prepare a pharmaceutical composition from a starting material such as a filament or powder that exhibits one or more favorable properties such as exhibiting a free-flowing property as an angle of repose, sufficient strength, sufficient stress, bend angle, diameter, viscosity, or Carr's Index. The co-crystal and the active pharmaceutical ingredient may comprise the active pharmaceutical ingredient and the co-former in a molar ratio from about 0.1 to about 10, from about 0.25 to about 4, or from about 0.5 to about 2. The molar ratio of the active pharmaceutical ingredient and the co-former is from about 0.1, 0.2, 0.25, 0.33, 0.5, 1, 2, 3, 4, 5, or 10. The amount of the composition which contains the co-crystal is greater than about 50%, 60%, 70%, 80%, 90%, 95%, 96%, 97%, 98%, or 99%. The pharmaceutical composition comprises from about 50%, 60%, 65%, 70%, 75%, 80%, 85%, 90%, 92.5%, 95%, 96%, 97%, 97.5%, 98%, 98.5%, 99%, 99.5%, to about 99.9%, or any range derivable therein of the co-crystal. The co-crystals may be formed between an active pharmaceutical ingredient and either an excipient or a second active pharmaceutical ingredient. These components of the co-crystals may have a pKₐ difference of less than 3, less than 2, less than 1.5, less than 1, less than 0.75, less than 0.5, or less than 0.25.

These compositions may exhibit one or more free-flowing properties such as having a flowability as measured by the angle of repose of less than 25. These compositions may exhibit a flowability as measured by the angle of repose of less than about 25, less than about 27.5, less than about 30, less than about 32.5, less than about 35, less than about 37.5, or less than about 40. The flowability may be from about 25 to about 40, or from about 25 to about 30. The flowability may be from about 2, 4, 5, 6, 8, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or any range derivable therein. The flowability of the pharmaceutical composition is measured by, the simplest method for the determination of the angle of repose is the “poured” angle. A funnel with a wide outlet is affixed at a distance of 10 cm above the bench, where a piece of paper is placed directly beneath the funnel. The granules are added while the funnel is closed. The contents flow through and collect on the paper. The diameter of the cone (D) and two opposite sides (l₁+l₂) are measured with rulers. The angle of repose (θ) is calculated from the equation arc cos[Dl(l₁+l₂)]. The relationship between flow properties and angle of repose has been established. When the angle of repose is less than 25 degrees, the flow is said to be excellent; on the other hand, if the angle of repose is more than 40 degrees, the flow is considered to be poor. These pharmaceutical compositions may be present as agglomerations and used in either a batch, semi-continuous, continuous manufacturing process. The active pharmaceutical ingredient may act as a binder between the absorbent particles within the pharmaceutical composition.

In other aspects, the present pharmaceutical compositions may exhibit a mean or average particle size distribution greater than 25 μm, greater than 50 μm, or greater than 60 μm. In some embodiments, the pharmaceutical compositions exhibit a mean or average particle size from about 25 μm to about 500 μm, 30 μm to about 400 μm, 35 μm to about 350 μm, 40 μm to about 300 μm, 50 μm to about 250 μm, 50 μm to about 200 μm, 50 μm to about 150 μm, 55 μm to about 125 μm, or from about 60 μm to about 100 μm. The mean or average particle size of the pharmaceutical composition comprises from about 25 μm, 30 μm, 35 μm, 40 μm, 45 μm, 50 μm, 55 μm, 60 μm, 65 μm, 70 μm, 75 μm, 80 μm, 85 μm, 90 μm, 95 μm, 100 μm, 105 μm, 110 μm, 115 μm, 120 μm, 125 μm, 150 μm, 175 μm, 200 μm, 250 μm, 300 μm, 350 μm, 400 μm, 450 μm, 500 μm, 600 μm, 700 μm, 800 μm, 900 μm, to about 1000 μm, or any range derivable therein. The mean or average particle size of the pharmaceutical composition may be determined by mesh analysis using a sonic sifter. The particle size distribution of the dried granules can also be determined by a dry laser diffraction technique or scanning electron microscopy.

Similarly, these compositions may exhibit a particle diameter, D₅₀, wherein 50% of the particles in the composition are larger than this particular particle size. The composition may have a D₅₀ of less than 100 μm, less than 75 μm, less than 60 μm, or less than 50 μm. Additionally, the composition may exhibit a particle diameter, D₉₀, wherein 90% of the particles in the composition are smaller than this particular particle size. The particles may have a D₉₀ wherein the D₉₀ is greater than 25 μm, greater than 40 μm, or greater than 50 μm. Alternatively, the D₉₀ may be less than 100 μm, less than 90 μm, less than 80 μm, or less than 75 μm. In some embodiments, the D₉₀ may be from about 10 μm to about 150 μm, from about 25 μm to about 100 μm, from about 50 μm to about 80 μm. The D₉₀ may be from about 10 μm, 25 μm, 30 μm, 40 μm, 45 μm, 50 μm, 55 μm, 60 μm, 65 μm, 70 μm, 75 μm, 80 μm, 85 μm, 90 μm, 10 μm, 100 μm, 105 μm, 110 μm, 120 μm, to about 125 μm, or any range derivable therein. The dry particle laser diffraction characterization methods were used to determine the particle size and distribution. A laser diffractometer with a disperser with the detection range from 0.1-875 μm was used to collect the particle size and distribution data. An optimal concentration of 0.1% was setup as the trigger condition and a feed rate of 50% and 3 bar.

In some aspects, the pharmaceutical composition may exhibit a Carr's Index is from about 5 to about 28, from about 5 to about 25, from about 5 to about 21, from about 5 to about 15, or from about 15 to about 25. The Carr's Index may be from about 2, 4, 5, 6, 7, 8, 9, 10, 11, 12, 13, 14, 15, 16, 17, 18, 19, 20, 21, 22, 23, 24, 25, 28, 30, 32, 35, 38, to about 40, or any range derivable therein. Carr's Index of the pharmaceutical composition may be determined by tapped density which is measured after a powder sample is subjected to mechanically tapping. The measurement procedure for bulk density and tapped density can be found in the US Pharmacopeia. Bulk density and tapped density can be used to calculate the Carr's compressibility index and Hausner ratio, which are measures of the propensity of a powder to flow and be compressed:

$$Compressibiliy index ( \% ) = \frac{tapped density - bulk density}{tapped density} \times 100 \\ Hausner ’ s ratio = \frac{Tapped density}{bulk density}$$

The bulk density of the composition may be less than 5 g/cm³, less than 4 g/cm³, less than 3 g/cm³, less than 2.5 g/cm³, less than 2.25 g/cm³, less than 2 g/cm³, less than 1.75 g/cm³, or less than 1.5 g/cm³. The bulk density may be in a range from about 0.25 g/cm³, 0.5 g/cm³, 0.75 g/cm³, 1 g/cm³, 1.25 g/cm³, 1.5 g/cm³, 1.75 g/cm³, 2 g/cm³, 2.25 g/cm³, 2.5 g/cm³, 3 g/cm³, 3.5 g/cm³, 4 g/cm³, 4.5 g/cm³, to about 5 g/cm³, or any range derivable therein. The bulk density were measured using a graduate cylinder by gently pass a quantity of powder sufficient to complete the test through a U.S. standard sieve #18 or smaller. The agglomerates formed during storage were break up before test. Approximately 100 g±1.0% (RSD) of the test sample (m) weighed were passed to a dry graduated cylinder of 250 ml (readable to 2 ml) without compacting, and read the unsettled apparent volume (Vo) to the nearest graduated unit. Calculate the bulk density in (g/cm³) using the formula m/Vo. The tapped density is measured by mechanically tapping a graduated measuring cylinder containing the powder sample. Powder samples were proceeded to a 250 ml graduated cylinder (readable to 2 ml) and a settling apparatus capable of producing 250±15 taps/min, and bulk volume (Vo) was determined using abovementioned methods. 10, 500 and 1250 taps on the same powder sample were conducted and the corresponding volumes V10, V500 and V1250 were recorded. (If the difference between V500 and V1250 is less than or equal to 2 ml, V1250 is the tapped volume. If the difference between V500 and V1250 exceeds 2 ml, repeat in increments such as 1250 taps, until the difference between succeeding measurements is less than or equal to 2 ml.) Calculate the tapped density (g/cm³) using the formula m/V_{f} in which V_{f} is the final tapped volume.

In some aspects, the present pharmaceutical composition may be exhibit compressibility that makes the composition useful for the production of pharmaceutical dosage forms such as oral forms like capsules or tablets. The pharmaceutical composition may also be used in a powder-based additive manufacturing application such as vat photopolymerization, material jetting, binder jetting, powder-bed fusion, material extrusion, directed energy deposition, or sheet lamination like fused deposition modeling, binder spraying, or selective laser sintering. These 3D printing platforms may be used in pharmaceutical manufacturing and patient-specific personalized therapy to produce on-demand pharmaceutical compositions.

A. Active Pharmaceutical Ingredient

In some embodiments, the active pharmaceutical ingredient is classified using the Biopharmaceutical Classification System (BCS), originally developed by G. Amidon, which separates pharmaceuticals for oral administration into four classes depending on their aqueous solubility and their permeability through the cell lining of the gastrointestinal tract. According to the BCS, drug substances are classified as follows: Class I-High Permeability, High Solubility; Class II-High Permeability, Low Solubility; Class III-Low Permeability, High Solubility; and Class IV-Low Permeability, Low Solubility.

In particular, typical BCS Class II that may be incorporated into the present pharmaceutical compositions include but are not limited to anti-infectious drugs such as Albendazole, Acyclovir, Azithromycin, Cefdinir, Cefuroxime axetil, Chloroquine, Clarithromycin, Clofazimine, Diloxanide, Efavirenz, Fluconazole, Griseofulvin, Indinavir, Itraconazole, Ketoconazole, Lopinavir, Mebendazole, Nelfinavir, Nevirapine, Niclosamide, Praziquantel, Pyrantel, Pyrimethamine, Quinine, and Ritonavir. Antineoplastic drugs such as Bicalutamide, Cyproterone, Gefitinib, Imatinib, and Tamoxifen. Biologic and Immunologic Agents such as Cyclosporine, Mycophenolate mofetil, Tacrolimus. Cardiovascular Agents such as Acetazolamide, Atorvastatin, Benidipine, Candesartan cilexetil, Carvedilol, Cilostazol, Clopidogrel, Ethylicosapentate, Ezetimibe, Fenofibrate, Irbesartan, Manidipine, Nifedipine, Nilvadipine, Nisoldipine, Simvastatin, Spironolactone, Telmisartan, Ticlopidine, Valsartan, Verapamil, Warfarin. Central Nervous System Agents such as Acetaminophen, Amisulpride, Aripiprazole, Carbamazepine, Celecoxib, Chlorpromazine, Clozapine, Diazepam, Diclofenac, Flurbiprofen, Haloperidol, Ibuprofen, Ketoprofen, Lamotrigine, Levodopa, Lorazepam, Meloxicam, Metaxalone, Methylphenidate, Metoclopramide, Nicergoline, Naproxen, Olanzapine, Oxcarbazepine, Phenytoin, Quetiapine Risperidone, Rofecoxib, and Valproic acid. Dermatological Agents such as Isotretinoin-Endocrine and Metabolic Agents such as Dexamethasone, Danazol, Epalrestat, Gliclazide, Glimepiride, Glipizide, Glyburide (glibenclamide), levothyroxine sodium, Medroxyprogesterone, Pioglitazone, and Raloxifene. Gastrointestinal Agents such as Mosapride, Orlistat, Cisapride, Rebamipide, Sulfasalazine, Teprenone, and Ursodeoxycholic Acid. Respiratory Agents such as Ebastine, Hydroxyzine, Loratadine, and Pranlukast. However, the skilled person will be well aware of other BCS class II drugs which can be used with the pharmaceutical compositions described herein.

Additionally, BCS class III drugs that may be incorporated into the present pharmaceutical compositions include but are not limited to cimetidine, acyclovir, atenolol, ranitidine, abacavir, captopril, chloramphenicol, codeine, colchicine, dapsone, ergotamine, kanamycin, tobramycin, tigecycline, zanamivir, hydralazine, hydrochlorothiazide, levothyroxine, methyldopa, paracetamol, propylthiouracil, i pyridostigmine, cloxacillin, thiamine, benzimidazole, didanosine, ethambutol, ethosuximide, folic acid, nicotinamide, nifurtimox, and salbutamol sulfate. However, the skilled person will be well aware of other BCS class III drugs which can be used with the pharmaceutical compositions described herein.

Additionally, BCS class IV drugs that may be incorporated into the present pharmaceutical compositions include but are not limited to hydrochlorothiazide, furosemide, cyclosporin A, itraconazole, indinavir, nelfinavir, ritonavir, saquinavir, nitrofurantoin, albendazole, acetazolamide, azithromycin, senna, azathioprine, chlorthalidone, BI-639667, rifabutin, paclitaxel, curcumin, etoposide, neomycin, methotrexate, atazanavir sulfate, Aprepitant, amphotericin B, amiodarone, or mesalamine. However, the skilled person will be well aware of other BCS class IV drugs which can be used with the pharmaceutical compositions described herein.

While the pharmaceutical compositions and methods described herein can be applied to any BCS class of drugs, BCS class II and IV are of interest for the pharmaceutical compositions described herein. Additionally, other API that are of specific consideration are those that are high melting point drugs such as a drug that has a melting point of greater than 200° C. Alternatively, the API used herein may have a melting point from about 25° C. to about 1,000° C., from about 100° C. to about 750° C., or from about 200° C. to about 500° C. In particular, the melting point may be greater than 200° C., 250° C., 300° C., 400° C., 500° C., 300° C., 700° C., 750° C., 800° C., 900° C., or 1,000° C.

In some aspects, the present methods may be used to formulate one or more poorly soluble API such as deferasirox, etravirine, indomethacin, posaconazole, and ritonavir. Etravirine is a neutral active pharmaceutical ingredient and may be used as a model for other neutral API. Deferasirox and indomethacin is a weak acid API and may be used as a model for other weak acid APIs. Posaconazole, itraconazole, and ritonavir are weak base APIs and may be used as models for other weak base APIs.

Suitable API may be any poorly water-soluble, biologically API or a salt, isomer, ester, ether or other derivative thereof, which include, but are not limited to, anticancer agents, antifungal agents, psychiatric agents such as analgesics, consciousness level-altering agents such as anesthetic agents or hypnotics, nonsteroidal anti-inflammatory agents (NSAIDS), anthelminthics, antiacne agents, antianginal agents, antiarrhythmic agents, anti-asthma agents, antibacterial agents, anti-benign prostate hypertrophy agents, anticoagulants, antidepressants, antidiabetics, antiemetics, antiepileptics, antigout agents, antihypertensive agents, anti-inflammatory agents, antimalarials, antimigraine agents, antimuscarinic agents, antineoplastic agents, antiobesity agents, antiosteoporosis agents, antiparkinsonian agents, antiproliferative agents, antiprotozoal agents, antithyroid agents, antitussive agent, anti-urinary incontinence agents, antiviral agents, anxiolytic agents, appetite suppressants, beta-blockers, cardiac inotropic agents, chemotherapeutic drugs, cognition enhancers, contraceptives, corticosteroids, Cox-2 inhibitors, diuretics, erectile dysfunction improvement agents, expectorants, gastrointestinal agents, histamine receptor antagonists, immunosuppressants, keratolytics, lipid regulating agents, leukotriene inhibitors, macrolides, muscle relaxants, neuroleptics, nutritional agents, opioid analgesics, protease inhibitors, or sedatives.

Non-limiting examples of the API may include 7-Methoxypteridine, 7-Methylpteridine, abacavir, abafungin, abarelix, acebutolol, acenaphthene, acetaminophen, acetanilide, acetazolamide, acetohexamide, acetretin, acrivastine, adenine, adenosine, alatrofloxacin, albuterol, alclofenac, aldesleukin, alemtuzumab, alfuzosin, alitretinoin, allobarbital, allopurinol, all-transretinoic acid (ATRA), aloxiprin, alprazolam, alprenolol, altretamine, amifostine, amiloride, aminoglutethimide, aminopyrine, amiodarone, amitriptyline, amlodipine, amobarbital, amodiaquine, amoxapine, amphetamine, amphotericin, amphotericin B, ampicillin, amprenavir, amsacrine, amylnitrate, amylobarbitone, anastrozole, anrinone, anthracene, anthracyclines, aprobarbital, arsenic trioxide, asparaginase, aspirin, astemizole, atenolol, atorvastatin, atovaquone, atrazine, atropine, atropine azathioprine, auranofin, azacitidine, azapropazone, azathioprine, azintamide, azithromycin, aztreonum, baclofen, barbitone, BCG live, beclamide, beclomethasone, bendroflumethiazide, benezepril, benidipine, benorylate, benperidol, bentazepam, benzamide, benzanthracene, benzathine penicillin, benzhexol, benznidazole, benzodiazepines, benzoic acid, bephenium hydroxynaphthoate, betamethasone, bevacizumab (avastin), bexarotene, bezafibrate, bicalutamide, bifonazole, biperiden, bisacodyl, bisantrene, bleomycin, bleomycin, bortezomib, brinzolamide, bromazepam, bromocriptine mesylate, bromperidol, brotizolam, budesonide, bumetanide, bupropion, busulfan, butalbital, butamben, butenafine, butobarbitone, butobarbitone (butethal), butoconazole, butoconazole nitrate, butylparaben, caffeine, calcifediol, calciprotriene, calcitriol, calusterone, cambendazole, camphor, camptothecin, camptothecin analogs, candesartan, capecitabine, capsaicin, captopril, carbamazepine, carbimazole, carbofuran, carboplatin, carbromal, carimazole, carmustine, cefamandole, cefazolin, cefixime, ceftazidime, cefuroxime axetil, celecoxib, cephradine, cerivastatin, cetrizine, cetuximab, chlorambucil, chloramphenicol, chlordiazepoxide, chlormethiazole, chloroquine, chlorothiazide, chlorpheniramine, chlorproguanil, chlorpromazine, chlorpropamide, chlorprothixene, chlorpyrifos, chlortetracycline, chlorthalidone, chlorzoxazone, cholecalciferol, chrysene, cilostazol, cimetidine, cinnarizine, cinoxacin, ciprofibrate, ciprofloxacin, cisapride, cisplatin, citalopram, cladribine, clarithromycin, clemastine fumarate, clioquinol, clobazam, clofarabine, clofazimine, clofibrate, clomiphene citrate, clomipramine, clonazepam, clopidogrel, clotiazepam, clotrimazole, clotrimazole, cloxacillin, clozapine, cocaine, codeine, colchicine, colistin, conjugated estrogens, corticosterone, cortisone, cortisone acetate, cyclizine, cyclobarbital, cyclobenzaprine, cyclobutane-spirobarbiturate, cycloethane-spirobarbiturate, cycloheptane-spirobarbiturate, cyclohexane-spirobarbiturate, cyclopentane-spirobarbiturate, cyclophosphamide, cyclopropane-spirobarbiturate, cycloserine, cyclosporin, cyproheptadine, cyproheptadine, cytarabine, cytosine, dacarbazine, dactinomycin, danazol, danthron, dantrolene sodium, dapsone, darbepoetin alfa, darodipine, daunorubicin, decoquinate, dehydroepiandrosterone, delavirdine, demeclocycline, denileukin, deoxycorticosterone, desoxymethasone, dexamethasone, dexamphetamine, dexchlorpheniramine, dexfenfluramine, dexrazoxane, dextropropoxyphene, diamorphine, diatrizoicacid, diazepam, diazoxide, dichlorophen, dichlorprop, diclofenac, dicumarol, didanosine, diflunisal, digitoxin, digoxin, dihydrocodeine, dihydroequilin, dihydroergotamine mesylate, diiodohydroxyquinoline, diltiazem, diloxamide furoate, dimenhydrinate, dimorpholamine, dinitolmide, diosgenin, diphenoxylate, diphenyl, dipyridamole, dirithromycin, disopyramide, disulfiram, diuron, docetaxel, domperidone, donepezil, doxazosin, doxazosin, doxorubicin (neutral), doxorubicin, doxycycline, dromostanolone propionate, droperidol, dyphylline, echinocandins, econazole, econazole nitrate, efavirenz, ellipticine, enalapril, enlimomab, enoximone, epinephrine, epipodophyllotoxin derivatives, epirubicin, epoetinalfa, eposartan, equilenin, equilin, ergocalciferol, ergotamine tartrate, erlotinib, erythromycin, estradiol, estramustine, estriol, estrone, ethacrynic acid, ethambutol, ethinamate, ethionamide, ethopropazine, ethyl-4-aminobenzoate (benzocaine), ethylparaben, ethinylestradiol, etodolac, etomidate, etoposide, etretinate, exemestane, felbamate, felodipine, fenbendazole, fenbuconazole, fenbufen, fenchlorphos, fenclofenac, fenfluramine, fenofibrate, fenoldepam, fenoprofen calcium, fenoxycarb, fenpiclonil, fentanyl, fenticonazole, fexofenadine, filgrastim, finasteride, flecamide acetate, floxuridine, fludarabine, fluconazole, fluconazole, flucytosine, fludioxonil, fludrocortisone, fludrocortisone acetate, flufenamic acid, flunanisone, flunarizine, flunisolide, flunitrazepam, fluocortolone, fluometuron, fluorene, fluorouracil, fluoxetine, fluoxymesterone, flupenthixol decanoate, fluphenthixol decanoate, flurazepam, flurbiprofen, fluticasone propionate, fluvastatin, folic acid, fosenopril, fosphenytoin sodium, frovatriptan, furosemide, fulvestrant, furazolidone, gabapentin, G-BHC (Lindane), gefitinib, gemcitabine, gemfibrozil, gemtuzumab, glafenine, glibenclamide, gliclazide, glimepiride, glipizide, glutethimide, glyburide, Glyceryltrinitrate (nitroglycerin), goserelin acetate, grepafloxacin, griseofulvin, guaifenesin, guanabenz acetate, guanine, halofantrine, haloperidol, hydrochlorothiazide, heptabarbital, heroin, hesperetin, hexachlorobenzene, hexethal, histrelin acetate, hydrocortisone, hydroflumethiazide, hydroxyurea, hyoscyamine, hypoxanthine, ibritumomab, ibuprofen, idarubicin, idobutal, ifosfamide, ihydroequilenin, imatinib mesylate, imipenem, indapamide, indinavir, indomethacin, indoprofen, interferon alfa-2a, interferon alfa-2b, iodamide, iopanoic acid, iprodione, irbesartan, irinotecan, isavuconazole, isocarboxazid, isoconazole, isoguanine, isoniazid, isopropylbarbiturate, isoproturon, isosorbide dinitrate, isosorbide mononitrate, isradipine, itraconazole, itraconazole, itraconazole (Itra), ivermectin, ketoconazole, ketoprofen, ketorolac, khellin, labetalol, lamivudine, lamotrigine, lanatoside C, lanosprazole, L-DOPA, leflunomide, lenalidomide, letrozole, leucovorin, leuprolide acetate, levamisole, levofloxacin, lidocaine, linuron, lisinopril, lomefloxacin, lomustine, loperamide, loratadine, lorazepam, lorefloxacin, lormetazepam, losartan mesylate, lovastatin, lysuride maleate, Maprotiline, mazindol, Meclizine, meclofenamic acid, medazepam, medigoxin, medroxyprogesterone acetate, mefenamic acid, Mefloquine, megestrol acetate, melphalan, mepenzolate bromide, meprobamate, meptazinol, mercaptopurine, mesalazine, mesna, mesoridazine, mestranol, methadone, methaqualone, methocarbamol, methoin, methotrexate, methoxsalen, methsuximide, methyclothiazide, methylphenidate, methylphenobarbitone, methyl-p-hydroxybenzoate, methylprednisolone, methyltestosterone, methyprylon, methysergide maleate, metoclopramide, metolazone, metoprolol, metronidazole, Mianserin, miconazole, midazolam, mifepristone, miglitol, minocycline, minoxidil, mitomycin C, mitotane, mitoxantrone, mofetilmycophenolate, molindone, montelukast, morphine, Moxifloxacin, nabumetone, nadolol, nalbuphine, nalidixic acid, nandrolone, naphthacene, naphthalene, naproxen, naratriptan, natamycin, nelarabine, nelfinavir, nevirapine, nicardipine, niclosamide, nicotin amide, nicotinic acid, nicoumalone, nifedipine, nilutamide, nimodipine, nimorazole, nisoldipine, nitrazepam, nitrofurantoin, nitrofurazone, nizatidine, nofetumomab, norethisterone, norfloxacin, norgestrel, nortriptyline, nystatin, oestradiol, ofloxacin, olanzapine, omeprazole, omoconazole, ondansetron, oprelvekin, ornidazole, oxaliplatin, oxamniquine, oxantelembonate, oxaprozin, oxatomide, oxazepam, oxcarbazepine, oxfendazole, oxiconazole, oxprenolol, oxyphenbutazone, oxyphencyclimine, paclitaxel, palifermin, pamidronate, p-aminosalicylic acid, pantoprazole, paramethadione, paroxetine, pegademase, pegaspargase, pegfilgrastim, pemetrexeddisodium, penicillamine, pentaerythritol tetranitrate, pentazocin, pentazocine, pentobarbital, pentobarbitone, pentostatin, pentoxifylline, perphenazine, perphenazine pimozide, perylene, phenacemide, phenacetin, phenanthrene, phenindione, phenobarbital, phenolbarbitone, phenolphthalein, phenoxybenzamine, phenoxybenzamine, phenoxymethyl penicillin, phensuximide, phenylbutazone, phenytoin, pindolol, pioglitazone, pipobroman, piroxicam, pizotifen maleate, platinum compounds, plicamycin, polyenes, polymyxin B, porfimersodium, posaconazole (Posa), pramipexole, prasterone, pravastatin, praziquantel, prazosin, prazosin, prednisolone, prednisone, primidone, probarbital, probenecid, probucol, procarbazine, prochlorperazine, progesterone, proguanil, promethazine, propofol, propoxur, propranolol, propylparaben, propylthiouracil, prostaglandin, pseudoephedrine, pteridine-2-methyl-thiol, pteridine-2-thiol, pteridine-4-methyl-thiol, pteridine-4-thiol, pteridine-7-methyl-thiol, pteridine-7-thiol, pyrantelembonate, pyrazinamide, pyrene, pyridostigmine, pyrimethamine, quetiapine, quinacrine, quinapril, quinidine, quinidine sulfate, quinine, quininesulfate, rabeprazole sodium, ranitidine rasburicase, ravuconazole, repaglinide, reposal, reserpine, retinoids, rifabutine, rifampicin, rifapentine, rimexolone, risperidone, ritonavir, rituximab, rizatriptan benzoate, rofecoxib, ropinirole, rosiglitazone, saccharin, salbutamol, salicylamide, salicylic acid, saquinavir, sargramostim, secbutabarbital, secobarbital, sertaconazole, sertindole, sertraline, simvastatin, sirolimus, sorafenib, sparfloxacin, spiramycin, spironolactone, stanolone, stanozolol, stavudine, stilbestrol, streptozocin, strychnine, sulconazole, sulconazole nitrate, sulfacetamide, sulfadiazine, sulfamerazine, sulfamethazine, sulfamethoxazole, sulfanilamide, sulfathiazole, sulindac, sulphabenzamide, sulphacetamide, sulphadiazine, sulphadoxine, sulphafurazole, sulphamerazine, sulpha-methoxazole, sulphapyridine, sulphasalazine, sulphinpyrazone, sulpiride, sulthiame, sumatriptan succinate, sunitinib maleate, tacrine, tacrolimus, talbutal, tamoxifen citrate, tamulosin, targretin, taxanes, tazarotene, telmisartan, temazepam, temozolomide, teniposide, tenoxicam, terazosin, terazosin, terbinafine, terbutaline sulfate, terconazole, terfenadine, testolactone, testosterone, tetracycline, tetrahydrocannabinol, tetroxoprim, thalidomide, thebaine, theobromine, theophylline, thiabendazole, thiamphenicol, thioguanine, thioridazine, thiotepa, thotoin, thymine, tiagabine, tibolone, ticlopidine, tinidazole, tioconazole, tirofiban, tizanidine, tolazamide, tolbutamide, tolcapone, topiramate, topotecan, toremifene, tositumomab, tramadol, trastuzumab, trazodone, tretinoin, triamcinolone, triamterene, triazolam, triazoles, triflupromazine, trimethoprim, trimipramine maleate, triphenylene, troglitazone, tromethamine, tropicamide, trovafloxacin, tybamate, ubidecarenone (coenzyme Q10), undecenoic acid, uracil, uracil mustard, uric acid, valproic acid, valrubicin, valsartan, vancomycin, venlafaxine, vigabatrin, vinbarbital, vinblastine, vincristine, vinorelbine, voriconazole, xanthine, zafirlukast, zidovudine, zileuton, zoledronate, zoledronic acid, zolmitriptan, zolpidem, and zopiclone.

In particular aspects, the API may be busulfan, taxane, or other anticancer agents; alternatively, itraconazole (Itra) and posaconazole (Posa) or other members of the general class of azole compounds. Exemplary antifungal azoles include a) imidazoles such as miconazole, ketoconazole, clotrimazole, econazole, omoconazole, bifonazole, butoconazole, fenticonazole, isoconazole, oxiconazole, sertaconazole, sulconazole and tioconazole, b) triazoles such as fluconazole, itraconazole, isavuconazole, ravuconazole, Posaconazole, voriconazole, terconazole, and c) thiazoles such as abafungin. Other API that may be used with this approach include, but are not limited to, hyperthyroid drugs such as carbimazole, anticancer agents like cytotoxic agents such as epipodophyllotoxin derivatives, taxanes, bleomycin, anthracyclines, as well as platinum compounds and camptothecin analogs. The following API may also include other antifungal antibiotics, such as poorly water-soluble echinocandins, polyenes (e.g., Amphotericin B and Natamycin) as well as antibacterial agents (e.g., polymyxin B and colistin), and anti-viral drugs. The API may also include a psychiatric agent such as an antipsychotic, anti-depressive agent, or analgesic and/or tranquilizing agents such as benzodiazepines. The API may also include a consciousness level-altering agent or an anesthetic agent, such as propofol. The present compositions and the methods of making them may be used to prepare a pharmaceutical composition with the appropriate pharmacokinetic properties for use as therapeutics.

B. Co-former

In some aspects, the present disclosure comprises one or more excipients formulated into pharmaceutical compositions as co-former with the active pharmaceutical ingredient to form a co-crystal. An “excipient” refers to pharmaceutically acceptable carriers that are relatively inert substances used to facilitate administration or delivery of an API into a subject or used to facilitate the processing of an API into drug formulations that can be used pharmaceutically for delivery to the site of action in a subject. Non-limiting examples of excipients include polymer-carriers, stabilizing agents, surfactants, surface modifiers, solubility enhancers, buffers, encapsulating agents, antioxidants, preservatives, nonionic wetting or clarifying agents, viscosity-increasing agents, and absorption-enhancing agents. In some embodiments, the pharmaceutical composition is substantially, essentially, or entirely free of any other excipient other than the co-former. In other embodiments, the composition comprises one or more excipients.

C. Excipient

In some aspects, the present disclosure comprises one or more excipients formulated into pharmaceutical compositions such as a pharmaceutically acceptable thermoplastic polymer. An “excipient” refers to pharmaceutically acceptable carriers that are relatively inert substances used to facilitate administration or delivery of an API into a subject or used to facilitate the processing of an API into drug formulations that can be used pharmaceutically for delivery to the site of action in a subject. Non-limiting examples of excipients include polymer-carriers, stabilizing agents, surfactants, surface modifiers, solubility enhancers, buffers, encapsulating agents, antioxidants, preservatives, nonionic wetting or clarifying agents, viscosity-increasing agents, and absorption-enhancing agents. In some embodiments, the pharmaceutical composition is substantially, essentially, or entirely free of any other excipient.

1. Fillers

In some aspects, the pharmaceutical composition may further comprise one or more inorganic or organic material that may be used to bulk up a composition to obtain an effective amount of the compound. The filler may be an inert inorganic or organic compound such as a salt like a calcium, magnesium, sodium, or potassium salt or a sulfate, chloride, or nitrate salt. Commonly used organic compounds include carbohydrates, sugars, and sugar derivatives such as mannitol, lactose, starch, or cellulose.

Furthermore, the pharmaceutical compositions described herein have a concentration of filler ranging from about 1% to about 99% w/w. In some embodiments, the amount of each absorbent is from about 1% to about 99% w/w, from about 25% to about 98% w/w, 50% to about 98% w/w, or 75% to about 97% w/w. The amount of each absorbent may be from about 10%, 25%, 30%, 35%, 40%, 45%, 50%, 55%, 60%, 65%, 70%, 75%, 80%, 85%, 90%, 92.5%, 95%, 96%, 97%, 98%, to about 99%, or any range derivable therein. In some embodiments, the pharmaceutical composition is substantially, essentially, or entirely free of any other fillers.

2. Other Excipients

In some aspects, the present disclosure provides pharmaceutical compositions that may further comprise one or more additional excipients. The excipients (also called adjuvants) that may be used in the presently disclosed compositions and composites, while potentially having some activity in their own right, for example, antioxidants, are generally defined for this application as compounds that enhance the efficiency and/or efficacy of the active pharmaceutical ingredient. It is also possible to have more than one active pharmaceutical ingredient in a given solution so that the particles formed contain more than one active pharmaceutical ingredient.

Any pharmaceutically acceptable excipient known to those of skill in the art may be used to produce the pharmaceutical compositions disclosed herein. Examples of excipients for use with the present disclosure include, lactose, glucose, starch, calcium carbonate, kaolin, crystalline cellulose, silicic acid, water, simple syrup, glucose solution, starch solution, gelatin solution, carboxymethyl cellulose, shellac, methyl cellulose, polyvinyl pyrrolidone, dried starch, sodium alginate, powdered agar, calcium carmelose, a mixture of starch and lactose, sucrose, butter, hydrogenated oil, a mixture of a quaternary ammonium base and sodium lauryl sulfate, glycerine and starch, lactose, bentonite, colloidal silicic acid, talc, stearates, and polyethylene glycol, sorbitan esters, polyoxyethylene sorbitan fatty acid esters, polyoxyethylene alkyl ethers, poloxamers (polyethylene-polypropylene glycol block copolymers), sucrose esters, sodium lauryl sulfate, oleic acid, lauric acid, vitamin E TPGS, polyoxyethylated glycolysed glycerides, dipalmitoyl phosphadityl choline, glycolic acid and salts, deoxycholic acid and salts, sodium fusidate, cyclodextrins, polyethylene glycols, polyglycolyzed glycerides, polyvinyl alcohols, polyacrylates, polymethacrylates, polyvinylpyrrolidones, phosphatidyl choline derivatives, cellulose derivatives, biocompatible polymers selected from poly(lactides), poly(glycolides), poly(lactide-co-glycolides), poly(lactic acid)s, poly(glycolic acid)s, poly(lactic acid-co-glycolic acid)s and blends, combinations, and copolymers thereof.

As stated, excipients and adjuvants may be used in the pharmaceutical composition to enhance the efficacy and efficiency of the active pharmaceutical ingredient in the pharmaceutical composition. Additional non-limiting examples of compounds that can be included are binders, carriers, cryoprotectants, lyoprotectants, surfactants, fillers, stabilizers, polymers, protease inhibitors, antioxidants, bioavailability enhancers, and absorption enhancers. The excipients may be chosen to modify the intended function of the active ingredient by improving flow, or bioavailability, or to control or delay the release of the API. Specific nonlimiting examples include sucrose, trehalose, Span 80, Span 20, Tween 80, Brij 35, Brij 98, Pluronic, sucroester 7, sucroester 11, sucroester 15, sodium lauryl sulfate (SLS, sodium dodecyl sulfate. SDS), dioctyl sodium sulphosuccinate (DSS, DOSS, dioctyl docusate sodium), oleic acid, laureth-9, laureth-8, lauric acid, vitamin E TPGS, Cremophor® EL, Cremophor® RH, Gelucire® 50/13, Gelucire® 53/10, Gelucire® 44/14, Labrafil®, Solutol® HS, dipalmitoyl phosphatidyl choline, glycolic acid and salts, deoxycholic acid and salts, sodium fusidate, cyclodextrins, polyethylene glycols, Labrasol®, polyvinyl alcohols, polyvinyl pyrrolidones, and tyloxapol.

The stabilizing carrier may also contain various functional excipients, such as: hydrophilic polymer, antioxidant, super-disintegrant, surfactant including amphiphilic molecules, wetting agent, stabilizing agent, retardant, similar functional excipient, or a combination thereof, and plasticizers including citrate esters, polyethylene glycols, PG, triacetin, diethyl phthalate, castor oil, and others known to those of ordinary skill in the art. Extruded material may also include an acidifying agent, adsorbent, alkalizing agent, buffering agent, colorant, flavorant, sweetening agent, diluent, opaquing agent, complexing agent, fragrance, preservative or a combination thereof.

Compositions with enhanced solubility may comprise a mixture of the active pharmaceutical ingredient and an additive that enhances the solubility of the active pharmaceutical ingredient. Examples of such additives include but are not limited to surfactants, polymer-carriers, pharmaceutical carriers, thermal binders, or other excipients. A particular example may be a mixture of the active pharmaceutical ingredient with a surfactant or surfactant, the active pharmaceutical ingredient with a polymer or polymers, or the active pharmaceutical ingredient with a combination of a surfactant and polymer carrier or surfactants and polymer-carriers. A further example is a composition where the active pharmaceutical ingredient is a derivative or analog thereof.

In some embodiments, the pharmaceutical compositions may further comprise one or more surfactants. Surfactants that can be used in the disclosed pharmaceutical compositions to enhance solubility include those known to a person of ordinary skill. Some particular non-limiting examples of such surfactants include but are not limited to sodium dodecyl sulfate, dioctyl docusate sodium, Tween 80, Span 20, Cremophor® EL or Vitamin E TPGS.

Solubility can be indicated by peak solubility, which is the highest concentration reached of a species of interest over time during a solubility experiment conducted in a specified medium at a given temperature. The enhanced solubility can be represented as the ratio of peak solubility of the agent in a pharmaceutical composition of the present disclosure compared to peak solubility of the reference standard agent under the same conditions. Preferably, an aqueous buffer with a pH in the range of from about pH 4 to pH 8, about pH 5 to pH 8, about pH 6 to pH 7, about pH 6 to pH 8, or about pH 7 to pH 8, such as, for example, pH 4.0, 4.5, 5.0, 5.5, 6.0, 6.2, 6.4, 6.6, 6.7, 6.8, 6.9, 7.0, 7.1, 7.2, 7.4, 7.6, 7.8, or 8.0, may be used for determining peak solubility. This peak solubility ratio can be about 2:1, 3:1, 4:1, 5:1, 6:1, 7:1, 8:1, 9:1, 10:1, 12:1, 15:1, 20:1, 25:1, 30:1, 35:1, 40:1, 45:1, 50:1, 55:1 or higher.

Compositions of the active pharmaceutical ingredient that enhance bioavailability may comprise a mixture of the active pharmaceutical ingredient and one or more pharmaceutically acceptable adjuvants that enhance the bioavailability of the active pharmaceutical ingredient. Examples of such adjuvants include but are not limited to enzyme inhibitors. Particular examples are such enzyme inhibitors include but are not limited to inhibitors that inhibit cytochrome P-450 enzyme and inhibitors that inhibit monoamine oxidase enzyme. Bioavailability can be indicated by the Cₘₐₓ or the AUC of the active pharmaceutical ingredient as determined during in vivo testing, where Cₘₐₓ is the highest reached blood level concentration of the active pharmaceutical ingredient over time of monitoring and AUC is the area under the plasma-time curve. Enhanced bioavailability can be represented as the ratio of Cₘₐₓ or the AUC of the active pharmaceutical ingredient in a pharmaceutical composition of the present disclosure compared to Cₘₐₓ or the AUC of the reference standard the active pharmaceutical ingredient under the same conditions. This Cₘₐₓ or AUC ratio reflecting enhanced bioavailability can be about 5:1, 6:1, 7:1, 8:1, 9:1, 10:1, 12:1, 15:1, 20:1, 25:1, 30:1, 35:1, 40:1, 45:1, 50:1, 55:1, 60:1, 65:1, 70:1, 75:1, 80:1, 85:1, 90:1, 95:1, 98:1, 99:1, 100:1 or higher.

In some aspects, the amount of the excipient in the pharmaceutical composition is from about 0.5% to about 20% w/w, from about 1% to about 10% w/w, from about 2% to about 8% w/w, or from about 3% to about 7% w/w. The amount of the excipient in the pharmaceutical composition comprises from about 0.5%, 1%, 1.5%, 2%, 2.5%, 3%, 3.5%, 4%, 4.5%, 5%, 5.5%, 6%, 6.5%, 7%, 7.5%, 8%, 9%, to about 10% w/w, or any range derivable therein, of the total pharmaceutical composition. In one embodiment, the amount of the excipient in the pharmaceutical composition is at 4% to 6% w/w of the total weight of the pharmaceutical composition.

### II. Additive Manufacturing Platforms

In some aspects, the pharmaceutical compositions described herein are processed in a final dosage form. The granules that are produced by the process may be further processed into a capsule or a tablet. Before formulation into a capsule or tablet, the granule may be further milled before being compressed into the capsule or tablet.

In other aspects, the pharmaceutical compositions described herein may also be used in an additive manufacturing platform. Some of the additive manufacturing platforms that may be used herein include 3D printing such as selective laser sintering or selective laser melting. Alternatively, a method such as stereolithography or fused deposition modeling may be used to obtain the final pharmaceutical composition. The pharmaceutical compositions described herein may be used these processes and exhibit a flowability as measured by the angle of repose of less than 25. The pharmaceutical composition may have a flowability of less than 25, less than 26, less than 27, less than 28, less than 29, less than 30, less than 32.5, less than 35, or less than 40.

These pharmaceutical compositions may be processed through laser sintering wherein a laser is aimed at a specific point on the pharmaceutical composition such that material is bound together to create a solid form. The laser is passed over the surface in a sufficient amount of time and sufficient location to produce the desired dosage form. The method relates to the use of the laser-based upon the power of the laser such as the peak laser power rather than the laser duration. The method often will make use of a pulsed laser. The laser used in these methods often is a high power laser such as a carbon dioxide laser. The process builds up the dosage form using cross-sections of the material through multiple scanning passes over the material. Additionally, the chamber of the 3D printer device may also be preheated to a temperature just below the melting point of the pharmaceutical composition such as the melting point of the composition as a whole or the active agent, the absorbent, or the surfactant. Furthermore, the method may be used without the need for a secondary feeder of material into the chamber of the device.

In some embodiments, the additive manufacturing techniques used in the present methods may include selective laser sintering 3D printing. This method may comprise use of a laser onto a composition that has been deposited into a chamber at particular locations. The laser acts to sinter the composition into a pharmaceutical composition. The formation of the final product is based upon the energy of the laser as well as the properties of the composition and the temperature of the composition and the chamber that the compositions are deposited into.

In the first part of the selective laser sintering process, the composition is deposited onto a surface in the chamber. The deposition of the composition may result in a layer, wherein the layer of the composition has a layer thickness (LT) from about 0.1 μm to about 100 mm, from about 1 μm to about 100 mm, from about 10 μm to about 100 mm, from about 50 μm to about 10 mm, from about 50 μm to about 1 mm, or from about 50 μm to about 100 μm. The layer thickness may be from about 0.1 μm, 1 μm, 10 μm, 25 μm, 30 μm, 35 μm, 40 μm, 45 μm, 50 μm, 55 μm, 60 μm, 65 μm, 70 μm, 75 μm, 80 μm, 85 μm, 90 μm, 95 μm, 100 μm, 105 μm, 110 μm, 115 μm, 120 μm, 125 μm, 130 μm, 135 μm, 140 μm, 145 μm, 150 μm, 175 μm, 200 μm, 250 μm, 300 μm, 350 μm, 400 μm, 450 μm, 500 μm, 600 μm, 700 μm, 750 μm, 800 μm, 900 μm, 1 mm, 5 mm, 10 mm, 25 mm, 50 mm, 75 mm, to about 100 mm.

The composition deposited into the surface in the chamber may be heated to a temperature, known as the surface temperature. This surface temperature may be used to provide additional energy to the composition to assist the preparation of the final pharmaceutical composition. The surface temperature may be a temperature from about 0° C. to about 500° C., from about 0° C. to about 250° C., from about 25° C. to about 250° C., from about 50° C. to about 175° C., or from about 75° C. to about 150° C. The surface temperature may be a temperature from about 0° C., 25° C., 50° C., 60° C., 70° C., 75° C., 80° C., 90° C., 100° C., 110° C., 120° C., 125° C., 130° C., 140° C., 150° C., 160° C., 170° C., 180° C., 190° C., 200° C., 210° C., 220° C., 230° C., 240° C., 250° C., 275° C., 300° C., 350° C., 400° C., 450° C., to about 500° C., or any range derivable.

Furthermore, the chamber may also be heated to a temperature known as the chamber temperature. The chamber temperature may be a temperature from about 0° C. to about 500° C., from about 0° C. to about 250° C., from about 25° C. to about 250° C., from about 50° C. to about 175° C., or from about 75° C. to about 150° C. The surface temperature may be a temperature from about 0° C., 25° C., 50° C., 60° C., 70° C., 75° C., 80° C., 90° C., 100° C., 110° C., 120° C., 125° C., 130° C., 140° C., 150° C., 160° C., 170° C., 180° C., 190° C., 200° C., 210° C., 220° C., 230° C., 240° C., 250° C., 275° C., 300° C., 350° C., 400° C., 450° C., to about 500° C., or any range derivable. In some embodiments, the chamber temperature is at least 1° C., at least 5° C., at least 10° C., at least 15° C., at least 20° C., at least 25° C., or at least 50° C. less than the surface temperature. The chamber temperature may be from 1° C. to about 50° C., 5° C. to about 25° C., 10° C. to about 25° C., or 10° C. to about 20° C. less than the surface temperature.

Once the composition has been deposited therein, the composition is exposed to a laser to sinter the composition to obtain the final pharmaceutical composition. The parameters of the laser may be used in obtaining a pharmaceutical composition with a co-crystal from the composition deposited in the chamber. The particular laser used by the process may further comprise a laser power from about 0.1 mW to about 25 W, from about 0.5 mW to about 10 W, from about 1 mW to about 1 W, or from about 1 mW to about 10 mW. The laser used herein may have a laser power from about 10 mW, 50 mW, 100 mW, 200 mW, 300 mW, 400 mW, 500 mW, 600 mW, 700 mW, 800 mW, 900 mW, 1 W, 5 W, 15 W, 20 W, to about 25 W, or any range derivable therein. The particular laser used may include a high power laser such as carbon dioxide laser, lamp or diode, pumped ND:YAG laser, and disk or fiber lasers. In some embodiment, a 2.3 watt solid diode 455 nm wavelength (visible light, bright blue) laser may be used. The laser used may emit light with a wavelength from about 50 nm to about 15,000 nm, from about 200 nm to about 11,000 nm, or from about 200 nm to about 1,000 nm. The wavelength may be 50 nm, 100 nm, 125 nm, 150 nm, 175 nm, 200 nm, 225 nm, 250 nm, 275 nm, 300 nm, 325 nm, 350 nm, 375 nm, 400 nm, 425 nm, 450 nm, 475 nm, 500 nm, 525 nm, 550 nm, 575 nm, 600 nm, 625 nm, 650 nm, 675 nm, 700 nm, 725 nm, 750 nm, 775 nm, 800 nm, 825 nm, 850 nm, 875 nm, 900 nm, 925 nm, 950 nm, 975 nm, 1,000 nm, 1,025 nm, 1,050 nm, 1,075 nm, 1,100 nm, 1,500 nm, 2,000 nm, 2,500 nm, 3,000 nm, 3,500 nm, 4,000 nm, 4,500 nm, 5,000 nm, 5,500 nm, 6,000 nm, 6,500 nm, 7,000 nm, 7,500 nm, 8,000 nm, 8,500 nm, 9,000 nm, 9,500 nm, 10,000 nm, 10,500 nm, 11,000 nm, 12,000 nm, 13,000 nm, 14,000 nm, to about 15,000 nm, or any range derivable therein. Furthermore, the laser used may have a specific beam size that indicates the size of the laser that strikes any particular point of the composition at a given time. The methods may further comprise using a laser with a beam size from about 0.1 μm to about 10 mm, from about 0.25 μm to about 1 mm, from about 1 μm to about 500 μm, or from about 2.5 μm to about 100 μm. The beam size may be a size from about 0.1 μm, 0.5 μm, 1 μm, 2.5 μm, 5 μm, 7.5 μm, 10 μm, 25 μm, 30 μm, 35 μm, 40 μm, 45 μm, 50 μm, 55 μm, 60 μm, 65 μm, 70 μm, 75 μm, 80 μm, 85 μm, 90 μm, 95 μm, 100 μm, 250 μm, 500 μm, 750 μm, 1 mm, to about 5 mm, or any range derivable therein.

The laser may be used to sinter the composition in a pattern. During the sintering process, the laser traces a pattern over the composition to prepare the final pharmaceutical composition. The pattern is prepared by passing the laser over the composition at a specific speed known as the laser speed (LS). The laser speed may be from about 0.1 mm/s to about 100,000 mm/s, from about 0.5 mm/s to about 50,000 mm/s, from about 1 mm/s to about 1,000 mm/s, or from about 25 mm/s to about 250 mm/s. The laser speed may be from about 0.1 mm/s, 0.25 mm/s, 0.5 mm/s, 0.75 mm/s, 1 mm/s, 5 mm/s, 10 mm/s 15 mm/s, 20 mm/s, 25 mm/s, 30 mm/s, 35 mm/s, 40 mm/s, 45 mm/s, 50 mm/s, 55 mm/s, 60 mm/s, 65 mm/s, 70 mm/s, 75 mm/s, 80 mm/s, 85 mm/s, 90 mm/s, 95 mm/s, 100 mm/s, 105 mm/s, 110 mm/s, 115 mm/s, 120 mm/s, 125 mm/s, 150 mm/s, 200 mm/s, 250 mm/s, 500 mm/s, 1,000 mm/s, 5,000 mm/s, 25,000 mm/s, 50,000 mm/s, to about 100,000 mm/s, or any range derivable therein. Furthermore, the laser may pass in a pattern over the composition in the surface of the chamber. The distances between the lines in the laser's pass are known as hatches. The distance between each successive laser pass is known as the hatch spacing. The methods used herein may include using a hatch spacing from about 5 mm to about 250 mm, from about 10 mm to about 200 nm, from about 10 mm to about 150 mm, or to about 10 to about 40 mm. The hatch spacing may be from about 1 mm, 5 mm, 10 mm, 15 mm, 17.5 mm, 20 mm, 21 mm, 22 mm, 22.5 mm, 23 mm, 24 mm, 25 mm, 26 mm, 27 mm, 27.5 mm, 28 mm, 29 mm, 30 mm, 32.5 mm, 35 mm, 37.5 mm, 40 mm, 45 mm, 50 mm, 60 mm, 70 mm, 80 mm, 90 mm, 100 mm, 125 mm, 150 mm, 175 mm, 200 mm, 225 mm, to about 250 mm, or any range derivable therein.

Finally, the combination of the chamber temperature and the surface temperature may be used to combine with the laser energy to provide sufficient energy to obtain the pharmaceutical composition. The amount of energy that the laser imparts into the pharmaceutical composition is calculated as the electron laser density. Electron laser density may be calculated using the following formula:

$$Electron Laser Density ( \frac{J}{{mm}^{3}} ) = \frac{Laser Power ( w )}{LS \times HS \times LT}$$

The electron laser density may be an amount of energy imparted from the laser from about 1 J/mm³ to about 500 J/mm³, from about 2.5 J/mm³ to about 500 J/mm³, from about 5 J/mm³ to about 250 J/mm³, from about 7.5 J/mm³ to about 100 J/mm³, or from about 7.5 J/mm³ to about 50 J/mm³. The electron laser density is from about 1 J/mm³, 1.5 J/mm³, 2 J/mm³, 2.5 J/mm³, 3 J/mm³, 3.5 J/mm³, 4 J/mm³, 4.5 J/mm³, 5 J/mm³, 5.5 J/mm³, 6 J/mm³, 6.5 J/mm³, 7 J/m³, 7.5 J/mm³, 8 J/mm³, 8.5 J/mm³, 9 J/mm³, 9.5 J/mm³, 10 J/mm³, 12.5 J/mm³, 15 J/mm³, 17.5 J/mm³, 20 J/mm³, 25 J/mm³, 50 J/mm³, 75 J/mm³, 100 J/mm³, 150 J/mm³, 200 J/mm³, 250 J/mm³, 300 J/mm³, 400 J/mm³, to about 500 J/mm³, or any range derivable therein.

In other embodiments, the compositions may be prepared using a fusion deposition modeling method. Fusion deposition modeling uses a filament which is then heated and extruded layer by layer upon a surface until the layers build the desired objects. Such deposition is produced from the extrusion by passing a nozzle from the extruder over the surface such that it deposits the pharmaceutical composition onto a surface in both the x, y, and z dimensions. As the first layer is deposited, the layer may be given some time to cool before the next layer is deposited. As described above for the laser sintering methods, the extruder may be passed over the composition and the filament or other material is deposited with a specific hatch spacing. The methods used herein may include using a hatch spacing from about 5 mm to about 250 mm, from about 10 mm to about 200 nm, from about 10 mm to about 150 mm, or to about 10 to about 40 mm. The hatch spacing may be from about 1 mm, 5 mm, 10 mm, 15 mm, 17.5 mm, 20 mm, 21 mm, 22 mm, 22.5 mm, 23 mm, 24 mm, 25 mm, 26 mm, 27 mm, 27.5 mm, 28 mm, 29 mm, 30 mm, 32.5 mm, 35 mm, 37.5 mm, 40 mm, 45 mm, 50 mm, 60 mm, 70 mm, 80 mm, 90 mm, 100 mm, 125 mm, 150 mm, 175 mm, 200 mm, 225 mm, to about 250 mm, or any range derivable therein. Alternatively, the methods used herein may include using a hatch spacing from about 5 mm to about 100 mm, from about 10 mm to about 75 nm, from about 10 mm to about 50 mm, or to about 10 to about 40 mm. The hatch spacing may be from about 1 mm, 5 mm, 10 mm, 15 mm, 17.5 mm, 20 mm, 21 mm, 22 mm, 22.5 mm, 23 mm, 24 mm, 25 mm, 26 mm, 27 mm, 27.5 mm, 28 mm, 29 mm, 30 mm, 32.5 mm, 35 mm, 37.5 mm, 40 mm, 45 mm, 50 mm, 60 mm, 70 mm, 80 mm, 90 mm, to about 100 mm, or any range derivable therein. Each of these passes over the object may be performed at a specific speed known as the print speed. The print speed may be from about 0.1 mm/s to about 100,000 mm/s, from about 0.5 mm/s to about 50,000 mm/s, from about 10 mm/s to about 1,000 mm/s, or from about 25 mm/s to about 250 mm/s. The print speed may be from about 0.1 mm/s, 0.25 mm/s, 0.5 mm/s, 0.75 mm/s, 1 mm/s, 5 mm/s, 10 mm/s 15 mm/s, 20 mm/s, 25 mm/s, 30 mm/s, 35 mm/s, 40 mm/s, 45 mm/s, 50 mm/s, 55 mm/s, 60 mm/s, 65 mm/s, 70 mm/s, 75 mm/s, 80 mm/s, 85 mm/s, 90 mm/s, 95 mm/s, 100 mm/s, 105 mm/s, 110 mm/s, 115 mm/s, 120 mm/s, 125 mm/s, 150 mm/s, 200 mm/s, 250 mm/s, 500 mm/s, 1,000 mm/s, 5,000 mm/s, 25,000 mm/s, 50,000 mm/s, to about 100,000 mm/s, or any range derivable therein.

The platform temperature may be a temperature from about 0° C., 25° C., 50° C., 60° C., 70° C., 75° C., 80° C., 90° C., 100° C., 110° C., 120° C., 125° C., 130° C., 140° C., 150° C., 160° C., 170° C., 180° C., 190° C., 200° C., 210° C., 220° C., 230° C., 240° C., 250° C., 275° C., 300° C., 350° C., 400° C., 450° C., to about 500° C., or any range derivable. In some embodiments, the methods contemplate a second platform temperature may be a temperature from about −125° C., −100° C., −90° C., −80° C., −75° C., −70° C., −60° C., −50° C., −40° C., −30° C., −25° C., −20° C., −10° C., 0° C., 10° C., 15° C., 20° C., 22.5° C., to about 25° C., or any range derivable.

This method can comprise the use of a filament. The filament has a diameter from about 0.5 mm to about 10 mm, from about 1 mm to about 7.5 mm, from about 1.5 mm to about 5 mm. The diameter of these filaments may be from about 0.25 mm, 0.5 mm, 0.75 mm, 1 mm, 1.25 mm, 1.5 mm, 1.75 mm, 2 mm, 2.25 mm, 2.5 mm, 3 mm, 3.5 mm, 4 mm, 4.5 mm, 5 mm, 6 mm, 7 mm, 7.5 mm, 8 mm, 9 mm, to about 10 mm, or any range derivable therein. The filaments that are used in these methods may exhibit some preferred strength or stress properties. Flexibility, brittleness and stiffness properties of the filaments were evaluated to represent the printability of the filaments. For Flexibility and brittleness evaluation, filament samples were cut into 50 mm in length. A TA-XT2 texture analyzer (Texture Technologies Corp, New York, USA) with a TA-95N 3-point bend apparatus were used to test the brittleness of the extruded filaments. 25 mm supporting gape and 1 mm blade were used, and blades moving speed is 10 mm/s until reach 15 mm below the samples. Each single formulation filaments were repeated 10 times. Breaking distance and load force/stress data were collected and analyzed. For stiffness analysis, filaments samples were placed on the solid platform and were cut into 5 mm in depth of the samples. The blade will cut into the sample for 35% shape change, and breaking stress/force data were collected. Each single formulation filaments were repeated 10 times. In particular, the filament may have a strength such that the forced needed to break the filament is greater than 1000 g, 2000 g, or 3000 g. Similarly, the filament may have a strength such that the forced needed to cut the filament is greater than 100 g, 200 g, or 300 g. Additionally, the stress needed to break the filament is greater than 5,000 g/mm², greater than 10,000 g/mm², or greater than 15,000 g/mm². Finally, the filaments used may have a bend angle such that the force needed to break the filament is greater than 10°, greater than 20° C., or greater than 30°. In addition, the force needed to cut into the filaments is greater than 1000 g, 2000 g, or 3000 g.

In other embodiments, the composition may be prepared using a binder spraying method. Using a binder spraying method, the method comprises applying a powder to a surface and then applying a liquid binder material to the powder such that the deposition of the next layer of powder sticks to the lower layer of powder. These methods similar to the selective laser sintering and fusion deposition methods requires the use of hatch spacing print speed. The methods used herein may include using a hatch spacing from about 5 mm to about 250 mm, from about 10 mm to about 200 nm, from about 10 mm to about 150 mm, or to about 10 to about 40 mm. The hatch spacing may be from about 1 mm, 5 mm, 10 mm, 15 mm, 17.5 mm, 20 mm, 21 mm, 22 mm, 22.5 mm, 23 mm, 24 mm, 25 mm, 26 mm, 27 mm, 27.5 mm, 28 mm, 29 mm, 30 mm, 32.5 mm, 35 mm, 37.5 mm, 40 mm, 45 mm, 50 mm, 60 mm, 70 mm, 80 mm, 90 mm, 100 mm, 125 mm, 150 mm, 175 mm, 200 mm, 225 mm, to about 250 mm, or any range derivable therein. Alternatively, the methods used herein may include using a hatch spacing from about 5 mm to about 100 mm, from about 10 mm to about 75 nm, from about 10 mm to about 50 mm, or to about 10 to about 40 mm. The hatch spacing may be from about 1 mm, 5 mm, 10 mm, 15 mm, 17.5 mm, 20 mm, 21 mm, 22 mm, 22.5 mm, 23 mm, 24 mm, 25 mm, 26 mm, 27 mm, 27.5 mm, 28 mm, 29 mm, 30 mm, 32.5 mm, 35 mm, 37.5 mm, 40 mm, 45 mm, 50 mm, 60 mm, 70 mm, 80 mm, 90 mm, to about 100 mm, or any range derivable therein. Each of these passes over the object may be performed at a specific speed known as the print speed. The print speed may be from about 1 mm/s to about 100,000 mm/s, from about 5 mm/s to about 50,000 mm/s, from about 10 mm/s to about 1,000 mm/s, or from about 25 mm/s to about 250 mm/s. The print speed may be from about 1 mm/s, 5 mm/s, 10 mm/s 15 mm/s, 20 mm/s, 25 mm/s, 30 mm/s, 35 mm/s, 40 mm/s, 45 mm/s, 50 mm/s, 55 mm/s, 60 mm/s, 65 mm/s, 70 mm/s, 75 mm/s, 80 mm/s, 85 mm/s, 90 mm/s, 95 mm/s, 100 mm/s, 105 mm/s, 110 mm/s, 115 mm/s, 120 mm/s, 125 mm/s, 150 mm/s, 200 mm/s, 250 mm/s, 500 mm/s, 1,000 mm/s, 5,000 mm/s, 25,000 mm/s, 50,000 mm/s, to about 100,000 mm/s, or any range derivable therein. These methods require the use of a liquid binder material. Some non-limiting examples of liquid binder materials include hydrocarbons such as: n-pentane, n-hexane, n-heptane, n-octane, n-nonane, n-decane, benzene, toluene, 2,2,4-trimethyl pentane, cyclohexane, 2,2,4-trimethylpentane, cyclohexane, ethylbenzene, ketones, acetone, methyl ethyl ketone, methyl isobutyl ketone, cyclohexanone, n-methyl-2-pyrrolidone, acetophenone; alcohols such as: methanol, ethanol, n-propanol, i-propanol, n-butanol, i-butanol, 2-butanol, n-amyl alcohol, i-amyl alcohol, cyclohexanol, n-octanol, ethanediol, diethylene glycol, 1,2-propanedioi; ethers such as: diethyl ether, diisopropyl ether, dibutyl ether, methyl tert butyl ether, 1,4-dioxane, tetrahydrofuran, esters, methyl acetate, ethyl acetate, isopropyl acetate, n-butyl acetate, cellosolve acetate, glycol ethers, propylene glycol methyl ether, ethylene glycol methyl ether, ethylene glycol ethyl ether, ethylene glycol monobutyl ether; chlorinated solvents such as: methylene chloride, chloroform, carbon tetrachloride, 1,2-dichloroethane, 1, i,1-trichloroethane, trichloroethylene, perchloroethylene, monochlorobenzene, miscellaneous solvents, dimethylformamide, dimethylacetamide, dimethylsulphoxide, sulfolane, carbon disulphide, acetic acid, aniline, nitrobenzene, morpholine, pyridine, 2-nitropropane, acetonitrile, furfuraldehyde, or phenol. These materials are often characterized by their viscosity wherein the liquid binder materials may have a viscosity from 0.1 mPa*s to 250 mPa*s, from about 0.25 mPa*s to 150 mPa*s, or from about 0.5 mPa*s to about 50 mPa*s. The viscosity of the liquid binder material may be from about 0.1 mPa*s, 0.25 mPa*s, 0.5 mPa*s, 1 mPa*s, 2.5 mPa*s, 5 mPa*s, 10 mPa*s, 25 mPa*s, 50 mPa*s, 75 mPa*s, 100 mPa*s, 125 mPa*s, 150 mPa*s, 175 mPa*s, 200 mPa*s, 225 mPa*s, to about 250 mPa*s, or any range derivable therein.

### III. Definitions

The use of the word “a” or “an”, when used in conjunction with the term “comprising” in the claims and/or the specification, may mean “one,” but it is also consistent with the meaning of “one or more,” “at least one,” and “one or more than one.” As used herein “another” may mean at least a second or more.

As used herein, the terms “active pharmaceutical ingredient”, “drug”, “pharmaceutical”, “active agent”, “therapeutic agent”, and “therapeutically active agent” are used interchangeably to represent a compound which invokes a therapeutic or pharmacological effect in a human or animal and is used to treat a disease, disorder, or other condition. In some embodiments, these compounds have undergone and received regulatory approval for administration to a living creature.

The use of the term “or” in the claims is used to mean “and/or” unless explicitly indicated to refer to alternatives only or the alternatives are mutually exclusive. As used herein “another” may mean at least a second or more.

The terms “compositions,” “pharmaceutical compositions,” “formulations,” “pharmaceutical formulations,” “preparations”, and “pharmaceutical preparations” are used synonymously and interchangeably herein.

“Treating” or treatment of a disease or condition refers to executing a protocol, which may include administering one or more drugs to a patient, to alleviate signs or symptoms of the disease. Desirable effects of treatment include decreasing the rate of disease progression, ameliorating or palliating the disease state, and remission or improved prognosis. Alleviation can occur before signs or symptoms of the disease or condition appearing, as well as after their appearance. Thus, “treating” or “treatment” may include “preventing” or “prevention” of disease or undesirable condition. In addition, “treating” or “treatment” does not require complete alleviation of signs or symptoms, does not require a cure, and specifically includes protocols that have only a marginal effect on the patient.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease. For example, treatment of cancer may involve, for example, a reduction in the size of a tumor, a reduction in the invasiveness of a tumor, a reduction in the growth rate of the cancer, or prevention of metastasis. Treatment of cancer may also refer to prolonging the survival of a subject with cancer.

“Subject” and “patient” refer to either a human or non-human, such as primates, mammals, and vertebrates. In particular embodiments, the subject is a human.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

“Pharmaceutically acceptable salts” means salts of compounds disclosed herein which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide, and calcium hydroxide. Acceptable organic bases include ethanolamine, diethanolamine, triethanolamine, tromethamine, N-methylglucamine, and the like. It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

The term “derivative thereof” refers to any chemically modified compound, wherein at least one of the compounds is modified by substitution of atoms or molecular groups or bonds. In one embodiment, a derivative thereof is a salt thereof. Salts are, for example, salts with suitable mineral acids, such as hydrohalic acids, sulfuric acid or phosphoric acid, for example, hydrochlorides, hydrobromides, sulfates, hydrogen sulfates or phosphates, salts with suitable carboxylic acids, such as optionally hydroxylated lower alkanoic acids, for example, acetic acid, glycolic acid, propionic acid, lactic acid or pivalic acid, optionally hydroxylated and/or oxo-substituted lower alkane dicarboxylic acids, for example, oxalic acid, succinic acid, fumaric acid, maleic acid, tartaric acid, citric acid, pyruvic acid, malic acid, ascorbic acid, and also with aromatic, heteroaromatic or araliphatic carboxylic acids, such as benzoic acid, nicotinic acid or mandelic acid, and salts with suitable aliphatic or aromatic sulfonic acids or N-substituted sulfamic acids, for example, methanesulfonates, benzenesulfonates, p-toluenesulfonates or N-cyclohexylsulfamates (cyclamates).

The term “amorphous” refers to a noncrystalline solid wherein the molecules are not organized in a definite lattice pattern. Alternatively, the term “crystalline” refers to a solid wherein the molecules in the solid have a definite lattice pattern. The crystallinity of the active pharmaceutical ingredient in the composition is measured by powder x-ray diffraction.

A “poorly soluble drug” refers to a drug that meets the requirements of the USP and BP solubility criteria of at least a sparingly soluble drug. The poorly soluble drug may be sparingly soluble, slightly soluble, very slightly soluble or practically insoluble. In a preferred embodiment, the drug is at least slightly soluble. In a more preferred embodiment, the drug is at least very slightly soluble. As defined by the USP and BP, a soluble drug is a drug which is dissolved from 10 to 30 part of solvent required per part of the solute, a sparingly soluble drug is a drug which is dissolved from 30 to 100 part of solvent required per part of the solute, a slightly soluble drug is a drug which is dissolved from 100 to 1,000 part of solvent required per part of the solute, a very slightly soluble drug is a drug which is dissolved from 1,000 to 10,000 part of solvent required per part of the solute, and a practically insoluble drug is a drug which is dissolved from 10,000 part of solvent required per part of solute. The solvent may be water that is at a pH from 1-7.5, preferably physiological pH.

As used in this specification and claim(s), the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps.

As used in this specification, the term “significant” (and any form of significance such as “significantly”) is not meant to imply statistical differences between two values but only to imply importance or the scope of the difference of the parameter.

Throughout this application, the term “about” is used to indicate that a value includes the inherent variation of error for the device, the method being employed to determine the value or the variation that exists among the study subjects or experimental studies. Unless another definition is applicable, the term “about” refers to ±10% of the indicated value.

As used herein, the term “substantially free of” or “substantially free” in terms of a specified component, is used herein to mean that none of the specified components has been purposefully formulated into a composition and/or is present only as a contaminant or in trace amounts. The total amount of all containments, by-products, and other material is present in that composition in an amount of less than 2%. The term “essentially free of” or “essentially free” is used to represent that the composition contains less than 1% of the specific component. The term “entirely free of” or “entirely free” contains less than 0.1% of the specific component.

The term “homogenous” is used to mean a composition in which the components are mixed in such a way that the components are uniformly distributed amongst the composition. In a preferred embodiment, the composition is uniformly distributed in such a manner that there are no regions of a single component that are greater than 1 μm or more preferably less than 0.1 μm. In one embodiment, the composition is so homogeneously mixed in such a manner that there are no atoms of the thermally conductive excipient are adjacent to another atom of the thermally conductive excipient.

The terms “substantially” or “approximately” as used herein may be applied to modify any quantitative comparison, value, measurement, or other representation that could permissibly vary without resulting in a change in the basic function to which it is related.

A temperature, when used without any other modifier, refers to room temperature, preferably 23° C. unless otherwise noted. An elevated temperature is a temperature that is more than 5° C. greater than room temperature; preferably more than 10° C. greater than room temperature.

The term “unit dose” refers to a formulation of the pharmaceutical composition such that the formulation is prepared in a manner sufficient to provide a single therapeutically effective dose of the active pharmaceutical ingredient to a patient in a single administration. Such unit dose formulations that may be used include but are not limited to a single tablet, capsule, or other oral formulations, or a single vial with a syringeable liquid or other injectable formulations. The resulting product can then undergo further downstream processing to create an intermediate product, such as granules, that can then be further formulated into a unit dose such as one prepared for oral delivery as tablets, capsules, three-dimensionally printed selective laser sintered (3DPSLS) or suspensions; pulmonary and nasal delivery; topical delivery as emulsions, ointments or creams; transdermal delivery; and parenteral delivery as suspensions, microemulsions or depot. In some forms, the final pharmaceutical composition that is produced is no longer a powder and is further produced as a homogenous final product. This final product has the capability of being processed into granules and being compressed or 3DPSLS into a final pharmaceutical unit dose form.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements and parameters.

Other objects, features, and advantages of the present disclosure will become apparent from the following detailed description. It should be understood, however, that the detailed description and the specific examples, while indicating preferred embodiments of the disclosure, are given by way of illustration only, since various changes and modifications within the spirit and scope of the disclosure will become apparent to those skilled in the art from this detailed description.

### IV. Examples

To facilitate a better understanding of the present disclosure, the following examples of specific embodiments are given. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure. In no way should the following examples be read to limit or define the entire scope of the disclosure.

### Example 1—Preparation of Formulations

### Example A

The ibuprofen (API, powders) with saccharin (co-former, powders) was physically mixed using a pestle and mortar.

TABLE 1
Formulation composition

| Formulation | Function | Melting point | Molar Ratio |
|---|---|---|---|
| Ibuprofen | API | 80.8° C. | 1:1 |
| Saccharin | Conformer | 230.8° C. | |

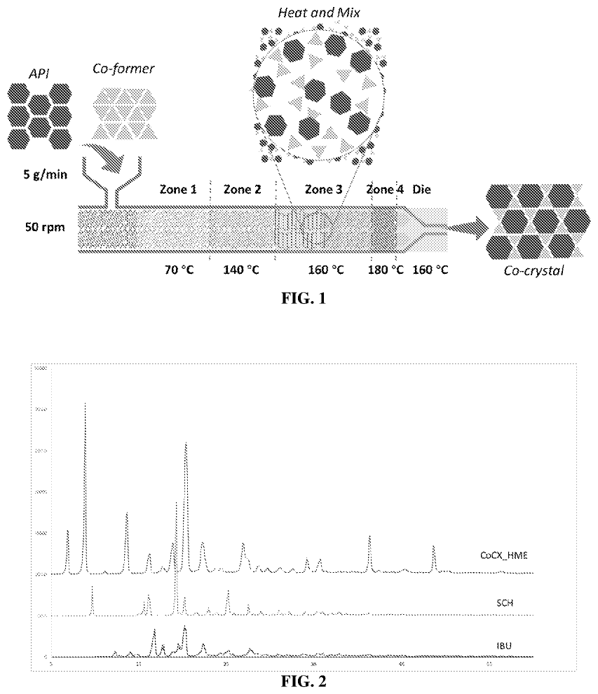

As shown in FIG. 1, the physically mixed ibuprofen and saccharin was fed into a 16 mm corotating twin-screw extruder at 5 g/min with four individual zones and temperature set was 70° C.-140° C.-160° C.-180° C.-160° C. from zone 1 to the die. The screw configuration was designed with 8 L/D kneading blocks in zone 3 and the screw speed was set at 50 RPM. The produced cocrystals were collected and characterized using various solid states characterization techniques including DSC, PLM, FT-IR, XRD, and Raman spectroscopy. This data is shown in FIG. 2.

### Example 2—Additive Manufacturing of the Cocrystal Loaded Devices

The manufactured cocrystals can be used for binder spraying platforms by mixing with 95% w/w filler, calcium sulfate and a 0.5% w/w hydroxypropyl cellulose solution was used as the binder solution. The tablets were designed with flexibly adjustable modulates, where the inner diameter (d_{In,n}) and outer diameter (D_{Out,n}) of ring-shaped modulates, and the diameter of the inner core (D_{Core}), height (Hₙ), and porosity (Pₙ) of each modulates can be adjusted for different drug release purposes, where the ‘n’ is the ring number labeled from inside to outside. A point worth noting here, is the tablets could be manufactured all at once (assembled), or separately then assembled manually depends on the purpose of use. Regardless of which way of manufacturing, the inner diameter (d_{In,n}) of each ring modulates is designed equally to the outer diameter (D_{Out,n-1}) of the next ring modulates inside it. In general, the diameter, height, and the number of layers have no limits within the swallowable range, as well as the printing techniques are accurate and precise enough for manufacturing. The porosity of each modulates could be 20%-100%

As an example shown in FIGS. 3A-3C, a batch of modulated medical devices was manufactured as assembled with three modulates, including a solid core (D_{Core}=3 mm, H_{Core}=5 mm, P=100%), a middle ring (d_{In,n},1=3 mm, D_{out,1}=6 mm, H_{Core}=5 mm, P=75%), and an out ring (d_{In,n}1=6 mm, D_{Out,1}=10 mm, H_{Core}=5 mm, P=50%).

### Example B

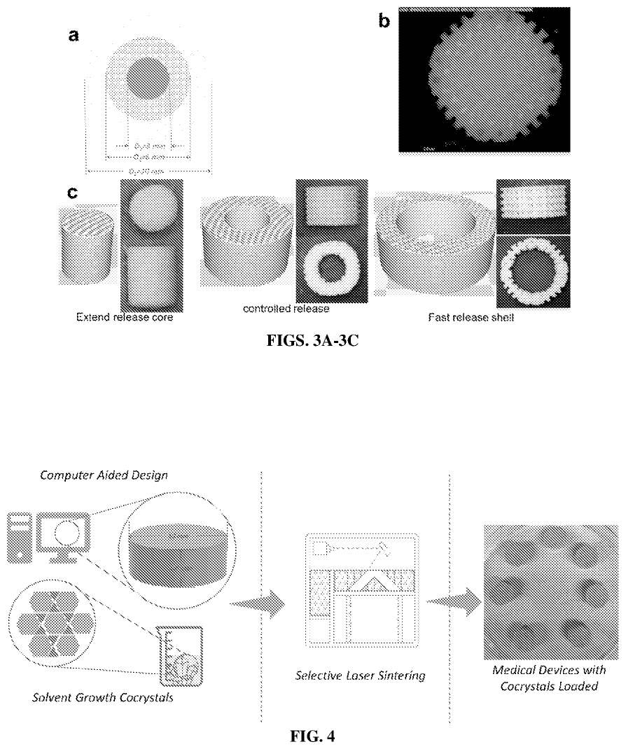

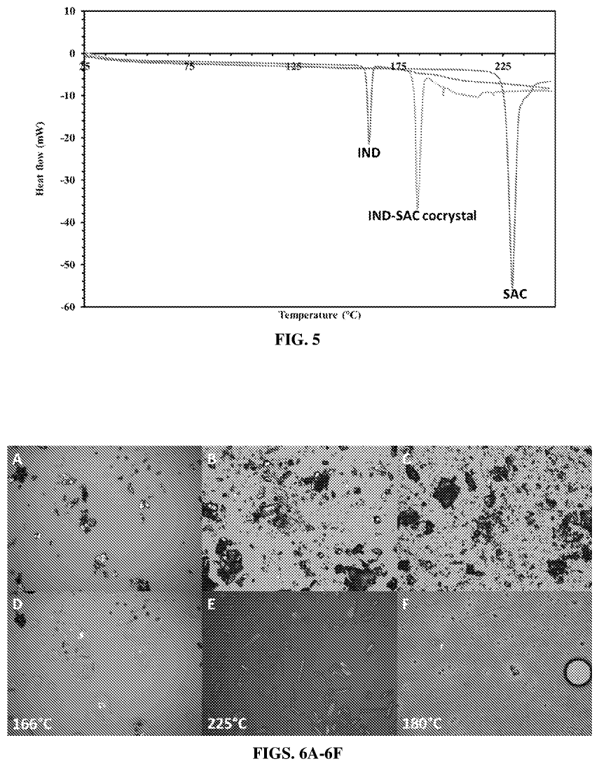

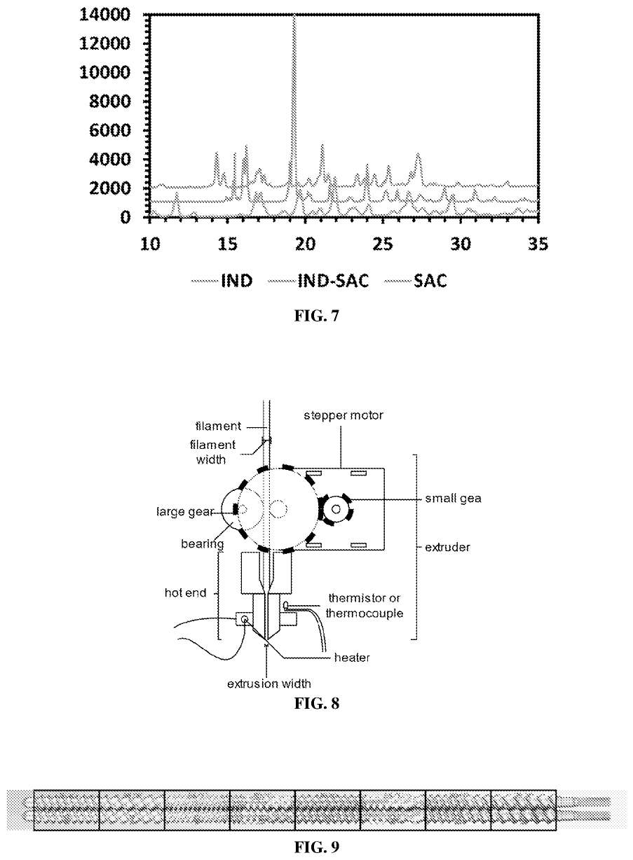

As the Example demonstrated in FIG. 4, Indomethacin-Saccharin (IND-SAC) cocrystals were prepared using a slow solvent evaporation method with acetone as the common solvent. The prepared solvents were physically mixed with 87% Kollidon VA64 and 3% candurin. This blend was used to produce 3D printed tablets containing IND-SAC cocrystals using selective laser sintering 3D printing techniques. For this process, the surface temperature was set to 110° C. and the chamber temperature was set to 90° C. The print speed was set to 50 mm/s and the hatch spacing was set to 25 microns. The results of this process are shown in FIGS. 5-8.

### Example C

Ibuprofen (IBU) and nicotinamide (NTM) were selected as the API and co-former for the cocrystal formulation, respectively. Soluplus was selected as the filler for the AM tablets, while Candurin Gold sheen was used as a sintering agent.

A Leistritz ZSE 12 HP-PH 12 mm twin screw corotating extruder with eight individual zones was used to extrude the combinations in order to form cocrystals. In this work, physical blends of the drug-coformer are fed into the extruder at room temperature (zone 1) and then conveyed (zone 2) to the first mixing zone (zone 3) to ensure a thorough mixing of the two ingredients. These are then conveyed (zone 4) to the second mixing zone (zone 5), where the temperature is set to melt one or both ingredients to obtain a molecular level mixing. Then convey, and vent zone (zone 6) let any accumulated moisture evaporate (if any) out of the barrel whereas the convey zone 7 and 8 are used to cool down the extrudates close to ambient temperature and convey materials out of the barrel (FIG. 9). The feeding rate is 6 g/mm, and the screw speed is 50 rpm.

i. Characterization of the Cocrystals

1. Differential Scanning Calorimetry (DSC)

A DSC Q20 equipment (TA® instruments, Delaware, USA) was used for the DSC analysis. 5-10 mg of pure API, co-former, physical mixtures, and extruded cocrystals were sealed in the standard aluminum pan and lids and ramped from 25 to complete melting temperature (depends on the samples) at a rate of 20° C./min. In all DSC experiments, ultra-purified nitrogen was used as the purge gas at a 50 mL/min flow rate. The data were collected and plotted as a plot of temperature (° C.) versus reverse heat flow (mW) using Excel software (Version 2007).

2. Powder X-Ray Diffraction (PXRD)

The solid state of pure API, co-former, physical mixtures, and extruded cocrystals were investigated using PXRD analysis using a benchtop PXRD instrument (MiniFlex, Rigaku Corporation, Tokyo, Japan). Briefly, the samples were scanned from a 2θ angle of 5 to 60 degrees, with a scan speed of 2 degrees/minute, scan step of 0.02 degrees, and the resultant scan resolution of 0.0025. The voltage was set at 45V, and the current was set at 15 mA during the scan process. The data were collected and plotted as a stacked plot of 2θ versus intensity using Excel software (Version 2007).

3. Hot-Stage Polarized Light Microscopy (PLM)

An Olympus BX53 polarizing photomicroscope (Olympus America Inc., Webster, TX, USA) equipped with Bertrand Lens was used to analyze the crystallinity of the pure API, co-former, physical mixtures, and extruded cocrystals. The samples were spread out evenly onto a glass slide. A coverslip was used to press and spread the samples as monolayer particles. The slide was placed on the microscope stage. All samples were observed under 10× magnification for birefringence property in crystalline substances. A QICAM Fast 1394 digital camera (QImaging, BC, Canada) with a 530 nm compensator (U-TP530, Olympus® corporation, Shinjuku City, Tokyo, Japan) was used to capture the images.

4. Fourier-Transform Infrared (FTIR) Spectroscopy

The pure API, co-former, physical mixtures, and extruded cocrystals were investigated using an Antaris analyzer with Nicolet iS50 series spectrophotometer (Thermal Fisher Scientific, Madison, WI, USA). Samples were positioned onto the face of the diamond crystal of the ATR unit, and the tip of the micrometer clamp was compressed onto the particles to allow adequate contact to get a characteristic spectrum. Backgrounds were collected using 32 scans for each 4 h during the FTIR measurements, while the spectrum of the sample was by scanning the specimens 32 times over a 750-4000 cm-1 range at a resolution of 4 cm-1 per sample.

5. Raman Spectroscopy

The pure API, co-former, physical mixtures, and extruded cocrystals were investigated using an Antaris analyzer (Thermal Fisher Scientific, Madison, WI, USA) equipped with a Raman scanning unit. Samples were positioned onto a 9-well sample holder. The samples spectrum was by scanning the specimens 32 times over a 300-4000 cm-1 range at a 4 cm-1 per sample.

ii. Preparation of the Cocrystal Loaded Tablets Via SLS

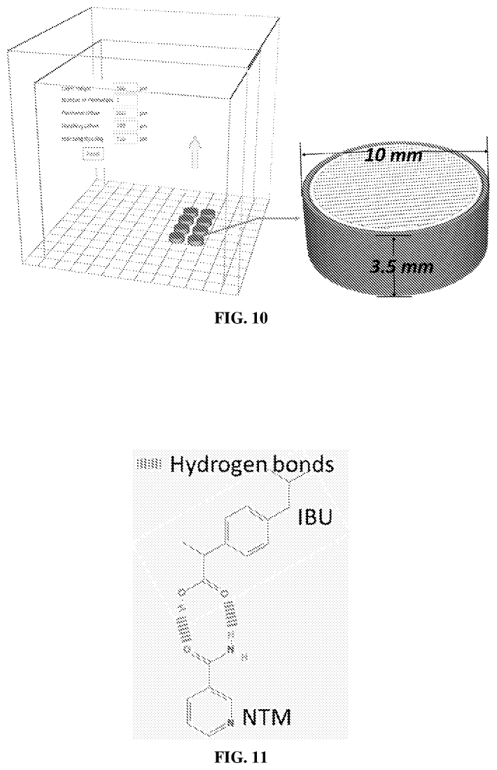

The model tablets were designed using Microsoft 3D Builder (Microsoft, Redmond, WA, USA) and saved in .stl format. Tablet was designed as cylinder shape with a diameter of 10 mm and thickness of 3.5 mm (FIG. 10). About 15% w/w of IBU-NTM cocrystal was mixed with 80% (w/w) of Soluplus and 5% (w/w) of Candurin Gold Sheen (a sintering agent) and then fabricated using a commercial Sintratec kit SLS 3D printer (Sintratec AG, Brugg, Switzerland). The following printer settings were found to produce the best tablets: a standard resolution with chamber and sintering temperature were both set at 35° C. The layer thickness was set at 0.1 mm, and hatching space was set at 0.12 mm. Two batches of the tablets were printed, where the laser scanning speed was set at 10 mm/s (SLS-10 tablets) and 1 mm/s (SLS-1 tablets).

Two kinds of directly compressed tablets were made as reference. A physical mixture of 15% (w/w) IBU: NTM=1:1 mixtures, 80% Soluplus, and 5% Candurin were used to prepare PM tablets, while the physical mixture of 15% (w/w) IBU-NTM cocrystals, 80% Soluplus, and 5% Candurin was used to prepare cocrystal physical mixture (CC-PM) tablets. Around 250 mg of mixtures of each formulation were compressed into tablets using a 10 mm die at 300 bars.

iii. Characterization of the Tablets

1. Assessment of Tablet Morphology

The diameters and thicknesses of the tablets were determined using a digital caliper, while the weight of the tablets was measured using a balance, and a DinoLite microscope camera was used to image the tablets.

2. Determination of Tablet Strength

A TA-XT2 analyzer (Texture Technologies Corp, New York, USA) coupled with a one-inch cylinder probe apparatus was used to assess the hardness of the printed tablets. The test speed was set at 5 mm/s, and the probe was stopped after reaching 5 mm after contacting the tablets. Each experiment was carried out in triplicates.

3. In Vitro Drug Release Study

Drug release from PM, CC-PM, SLS-10, and SLS-1 tablets was determined using a United States Pharmacopeia (USP)-II dissolution apparatus. Dissolution tests were conducted as per the U.S. Pharmacopeial standards using Simulated Intestinal Fluid^{TS} (without pancreatin) (pH 6.8), which represents the small intestinal fluid of humans. Each experiment was carried out in triplicate using 900 mL of the dissolution medium at 37±0.5° C. for 3 h. The paddle speed was set at 50 rpm. Samples were taken at 2.5, 5, 10, 15, 30, 60, 120, and 180 min and analyzed. The amount of released IBU was determined by HPLC at 253 nm.

iv. Results and Discussion

1. Characterization of the Cocrystals

The IBU-NTM were premixed and feed into the extruder at room temperature while completely melting at zone 5, and particles or granules were discharged from zone 8. As shown in FIG. 11 above, the IBU and NTM can form cocrystals by forming a hydrogen bond between the amine and carboxyl groups, confirmed via solid states analysis.

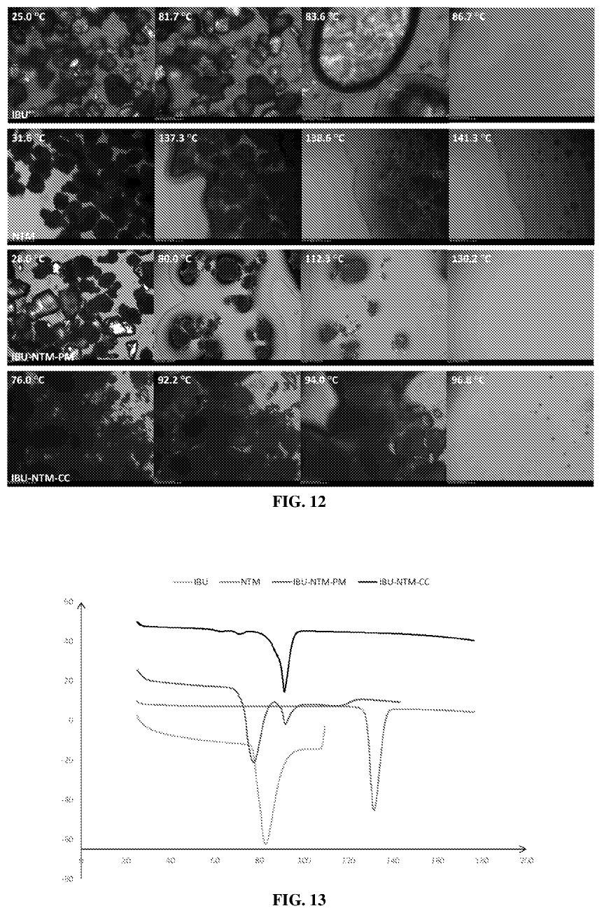

PLM figures (FIG. 12) showed that IBU and NTM melt around 84° C. and 139° C., respectively. The physical mixture of the IBU-NTM starts melting at around 80° C., which is because of low melting point of IBU, while NTM melts or dissolve into the molten IBU before reaching 130° C. This is mainly because of the interaction between the two molecules' function groups, which results in the adequate miscibility of two ingredients. Small amounts of particles start to melt at around 90° C., which is mainly due to the existence of free IBU in the physical mixture. The cocrystals showed a single step of melting with an onset of 94° C. instead of two separate melting events shown in the physical mixtures, which potentially proved the formation of the cocrystals.

DSC figures (FIG. 13) can cross-verify the observation from the PLM figures, where IBU and NTM have a melting peak of around 82.87° C. and 131.34° C., respectively. And two isolated melting peaks can be observed in the physical mixture trace, where the first peak corresponds to the melting of IBU and the second one indicates the melting of NTM. The cocrystals showed a melting peak of 91.29° C.

PXRD (FIG. 14) also complement the finding from the thermal analsys. The diffractogram of IBU showed characteristic diffraction at around 20 of 16.80, 17.68, 19.48, 20.24, 22.32, and 27.68°, and NTM showed peaks around 20 of 15.00, 22.30, 23.12, and 27.50°. The cocrystals showed characteristic peaks around 20 of 16.50, 17.36, 18.10, 25.12, and 28.12°. An additional new peak at ˜10 2θ position is evident for the cocrystal formulation. This particular peak is not present in any of the bulk components nor in the physical mixture which indicates the formation of new crystal forms other than the IBU or NTM.

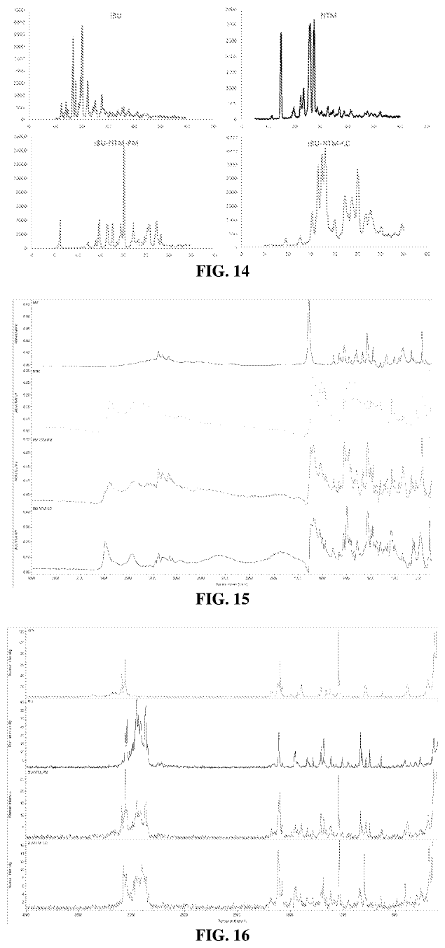

FTIR (FIG. 15) showed the intermolecular interactions of the cocrystals. The carboxyl group of IBU can be identified around wavenumbers of 1718 and 930 cm⁻¹, while the amine group of NTM can be identified around the wavenumber range of 1540-1450 cm⁻¹. The existence of the hydrogen bond in cocrystals results in the shift of the abovementioned peaks to the lower wavenumbers.

Raman (FIG. 16) showed the intramolecular movements of the cocrystals. The N-H rocking movement of amine can be identified at the Raman shift of 1049 cm⁻¹ in NTM and physical mixtures, while it shifts to lower wavenumbers (1035 cm⁻¹) in the IBU-NTM cocrystals. The asymmetrical stretching of can be identified around 3035-3110 cm⁻¹ in IBU, NTM, and physical mixtures, while it is broadened to 3003-3129 cm⁻¹ in the IBU-NTM cocrystals because of hydrogen bonding.

v. Characterization of the Tablets

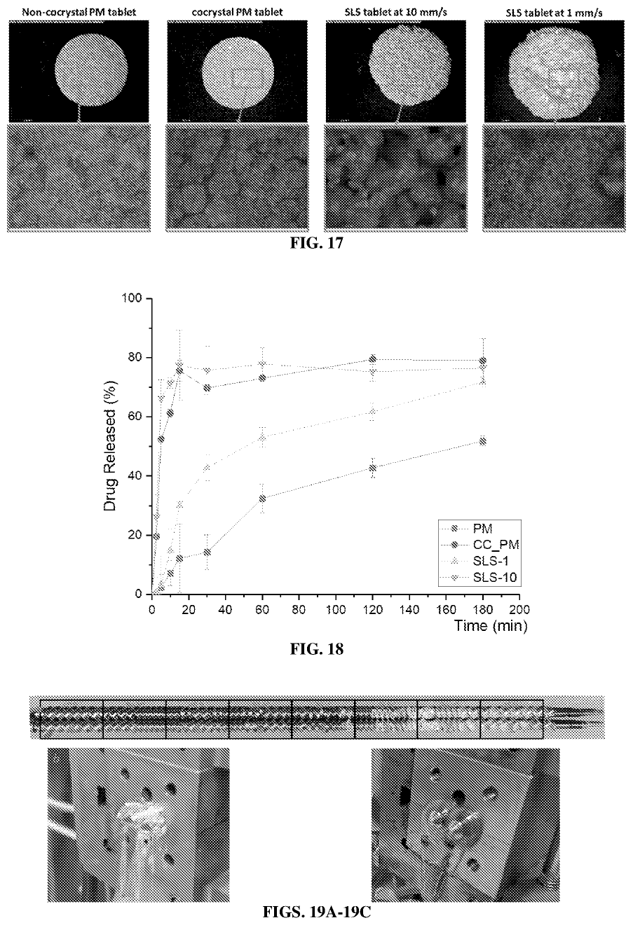

As shown in FIG. 17, the IBU-NTM cocrystal-loaded tablets were successfully prepared via SLS printing. The directly compressed tablets showed a more uniform and consistent appearance than the SLS printed tablets, where all the particles were physically compressed. However, the SLS tablets showed more uniform drug distribution than the PM tablets, where the drug ingredients can be identified in the bottom left corner picture of FIG. 17. The 10 mm/s SLS tablets showed that Soluplus were slightly softened and sintered together and formed the tablets, while the 1 mm/s SLS tablets showed that all the particles were sintered together and formed the tablets.

Additionally, the PM and SLS-10 tablets were extremely fragile because the tablets can be easily broken with hands, and particles were easy to lose. The CC-PM tablets showed more robust characteristics compared to the other two kinds of tablets, which indicates the excellent compressibility of the cocrystals. The longer sintering time let the particulates sinter more inseparable, which makes the SLS-ltablets stronger than the SLS-10 tablets.

As listed in Table 2, the PM tablets showed good reproducibility where all the dimension, weight, and hardness variations are less than 2.65%. However, the SLS tablets showed relatively higher standard deviations (>5%), which is mainly because of the loose structure of the tablets. In addition, the tablets were designed as 10×3.5 mm, but the SLS-10 and SLS-1 showed 13.83% and 9.45% of the variation in diameter compared to the digital design, respectively. The SLS-1 tablets showed more minor variations than the SLS-10 tablets, indicating that the slower scanning speed will lead to a better quality.

TABLE 2
List of the dimensions, weight, density, and hardness of the tablets prepared
using different techniques (mean ± S.D., n = 6).

| | D | H | M | ρ | Hardness |
|---|---|---|---|---|---|
| | (mm) | (mm) | (mg) | (mg/mm3) | (N) |
| PM | 10.00 ± 0.01 | 3.50 ± 0.01 | 249.67 ± 1.75 | 0.9081 ± 0.01 | 94.57 ± 3.51 |
| PM-CC | 10.00 ± 0.01 | 3.23 ± 0.05 | 249.17 ± 1.60 | 0.9843 ± 0.02 | 149.38 ± 2.65 |
| SLS-10 | 11.38 ± 0.61 | 3.41 ± 0.17 | 190.17 ± 16.44 | 0.5544 ± 0.09 | 67.97 ± 6.71 |
| SLS-1 | 10.95 ± 0.49 | 3.47 ± 0.09 | 186.67 ± 7.42 | 0.5751 ± 0.06 | 125.44 ± 15.43 |

vi. In Vitro Drug Release Studies

During the dissolution studies, all the tablets were observed floating at the beginning which might be due to low tablet densities. Interestingly, none of the tablets disintegrated in the first 60 min of the dissolution studies. As shown in FIG. 18, the PM tablets showed slower drug release rates, representing the release of IBU from the conventional tablets. The CC-PM and SLS-10 tablets showed relatively faster drug release rates which indicate the formation of the cocrystals can improve the dissolution of the poorly water-soluble drugs. Additionally, the existence of the hydrophilic polymers within the system can also maintain a thermodynamic metastable condition for poorly water-soluble drugs as well. The SLS-1 tablets showed an extended drug release rate than the SLS-10 tablets, which might be due to the longer sintering time that made the tablet stronger and more condensed than the SLS-10 tablets.

Ibuprofen-Nicotinamide Cocrystals Loaded Tablets Manufactured by FDM

i. Formulation

IBU and NTM were selected as the API and co-former for the cocrystal formulation. Ultimaker Natutral PVA filaments were used to prepare the blank tablets. A Leistritz ZSE 12 HP-PH 12 mm twin screw corotating extruder with eight individual zones was used to extrude the combinations in order to form cocrystals. In this work, the materials are fed into the extruder at the ambient temperature (zone 1) and then conveyed (zone 2) to the first mixing zone (zone 3) to assure the thorough mixing of the two ingredients. Then conveyed (zone 4) to the second mixing zone (zone 5), where the temperature was at 80° C. to ensure a molecular level mixing. All extruded materials are then discharged out of the barrel (FIG. 11A & 11C). The feeding rate was set at 6 g/min, and the screw speed was 50 rpm.

Here, molten extrudates were collected and then cooled to room temperature in a vacuum desiccator. After solidification, the extrudates were milled for further analysis. The same characterization was done on these below.

ii. Preparation of the Cocrystal Loaded Tablets Via FDM

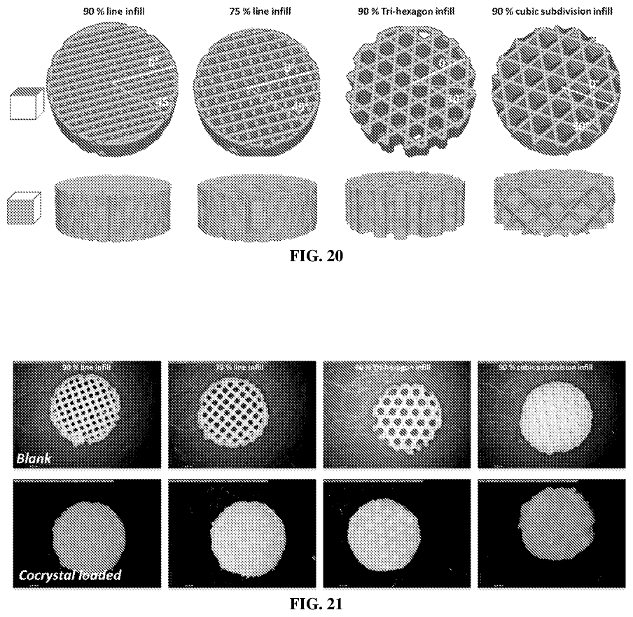

All blank tablets without cocrystals were designed using 3D builder software (version 18.0.1931.0, Microsoft Corporation) in a cylinder shape (height, 3.5 mm; and diameter, 10 mm). As shown in the FIG. 20, the models were then sliced to different tablets with designed infill patterns and infill densities using CURA software (version 4.6.2, Ultimaker). An Ultimaker S3 printer with a 0.4 mm printing nozzle was used to produce the designed tablets. All tablets were printed using pharmaceutical PVA or other hydrophilic/hydrophobic polymeric filaments prepared in our lab and under the same printing conditions. The printing and building bed temperatures were set at 180° C., and 60° C., respectively, and the blank tablets were printed at a printing speed of 50 mm/s.

After collecting the molten extrudates and before cooling down and recrystallizing, the printed blank tablets were submerged into the molten materials to load the cocrystals. After the complete solidification, tablets were collected and extra cocrystal outside the dimension of the tablet was manually cleaned.

iii. Characterization of the Tablets

1. Assessment of Tablet Morphology

The diameters and thicknesses of the tablets were determined using a digital caliper, while the weight of the tablets was measured using a balance, and a DinoLite microscope camera was used to image the tablets.

2. Determination of Tablet Strength

A TA-XT2 analyzer (Texture Technologies Corp, New York, USA) coupled with a one-inch cylinder probe apparatus was used to assess the hardness of the printed tablets. The test speed was set at 5 mm/s, and the probe was stopped after reaching 5 mm after contacting the tablets. Because of the specific infill patterns, tablets of 75% and 90% line infill were tested in 0° and 45°, while 90% tri-hexagon and cubic subdivision infill tablets were tested in 0° and 300 (FIG. 5). Each experiment was carried out in triplicates.

3. In Vitro Drug Release Study

Drug release from cocrystal-loaded tablets was determined using a United States Pharmacopeia (USP)-II dissolution apparatus. Dissolution tests were conducted as per the U.S. Pharmacopeial standards using Simulated Intestinal Fluid^{TS} (without pancreatin) (pH 6.8), which represents the small intestinal fluid of humans. Each experiment was carried out in triplicate using 900 mL of the dissolution medium at 37±0.5° C. for 3 h. The paddle speed was set at 50 rpm. Samples were taken at 2.5, 5, 10, 15, 30, 60, 120, and 180 min and analyzed. The amount of released IBU was determined by HPLC at 253 nm.

iv. Results and Discussion

1. Characterization of the Cocrystals

Because the same IBU-NTM cocrystals used in the current investigation, the results and discussions are the same described above.

I. Characterization of the Tablets

As shown in FIG. 21, the IBU-NTM cocrystal-loaded FDM tablets were successfully obtained. The cocrystals filled the porous structures of the FDM printed tablets. As listed in Table 2, the dimensions of the cocrystal loaded tablets were relatively larger than the blank tablets, which was mainly due to the cocrystals' loading. The varied drug content of the cocrystals-loaded tablets is directly affected by the weight, which is fundamentally influenced by the 3-dimensional designs. As shown in Table 2, the drug loading (DL, % w/w) ratio of different designed tablets was varied from 47.07% (T_{CC-90Cubic}) to 63.50% (T_{CC-75L}). As expected, the T_{CC-75L} has a relatively large drug loading than T_{90L}, which indicates that the lower the infill density, the more the cocrystals loaded. However, the T_{CC-90Cubic} has a lower Drug loading ratio than other designs, mainly due to the specific 3D structures.

TABLE 2
List of the dimensions, weight, density, and hardness
of the tablets prepared with different designs.

| | D (mm) | H (mm) | W (mg) | ρ (mg/mm³) | DL (%) | H 0° | H 45° | **CV₁ (%) |
|---|---|---|---|---|---|---|---|---|
| T_{90 L} | 10.01 | 3.50 | 135.33 | 0.4912 | 51.89 | 474.28 | 417.50 | 11.97 |
| T_{CC-90 L} | 10.22 | 3.65 | 281.29 | 0.9392 | | 484.5 | 427.33 | 11.80 |
| *CV (%) | 2.05 | 4.11 | 51.89 | 47.70 | | 2.11 | 2.30 | −/− |
| T_{75 L} | 10.00 | 3.50 | 115.83 | 0.4211 | 63.50 | 429.57 | 378.38 | 11.92 |
| T_{CC-75 L} | 10.82 | 3.64 | 317.33 | 0.9467 | | 451.31 | 381.17 | 15.54 |
| CV (%) | 7.58 | 3.85 | 63.50 | 55.52 | | 4.82 | 0.73 | −/− |
| T_{90Tri} | 10.02 | 3.50 | 138.00 | 0.4999 | 54.62 | 440.11 | 375.72 | 14.63 |
| T_{CC-90Tri} | 10.85 | 3.64 | 304.09 | 0.9045 | | 466.06 | 391.16 | 16.07 |
| CV (%) | 7.65 | 3.85 | 54.62 | 44.72 | | 5.57 | 3.95 | −/− |
| T_{90Cubic} | 10.00 | 3.51 | 155.10 | 0.5635 | 47.07 | 478.22 | 450.13 | 5.87 |
| T_{CC-90Cubic} | 10.21 | 3.64 | 293.01 | 0.9861 | | 485.04 | 458.28 | 5.52 |
| CV (%) | 2.06 | 3.57 | 47.07 | 42.86 | | 1.41 | 1.78 | −/− |

*CV: coefficient of the variation between the cocrystal loaded tablets and blank tablets, %.
**CV₁: coefficient of the variation between the tablets tested under different angles, %.

It is easy to observe that the tablets' hardness depended on the orientation under the texture analyzer. As shown in FIG. 20, the mechanical properties should be orientation depending on the 3D printed tablets due to the specific design. In the current investigation, the T_{90L} and T_{75L} tablets were printed using the “line” fill pattern, where the fill patterns were parallel lines within the same layer, and there is an angle of 90° between two adjacent layers. For the hardness test, two different orientations, 0°, and 45° were tested, where the probe movement aligned with the infill printing patterns or the diagonal direction of the crossed printing patterns, respectively. As expected, for most of the tablets, the variation between two different orientations was relatively high (CV₁>11.80%), was more condensed compared to the other tablets. Similar results were also observed from the T_{90Tri} and T_{90cubic} tablets, where the fill pattern were combinations of hexagons and triangles. Besides, due to the layered structures, the T_{90cubic} tablets' mechanical properties are more robust which might be because of the more supportive structures with each layer.

As listed in Table 2, the cocrystals loaded tablets showed slightly stronger mechanical properties than the blank tablets, where the variation between blank and cocrystal loaded tablets was small (CV<5.57%). This might be because a load of cocrystals has not reached the maximum capacity of blank tablets and has limited influence on the mechanical properties, which indicates more cocrystals could be loaded.

2. In Vitro Drug Release

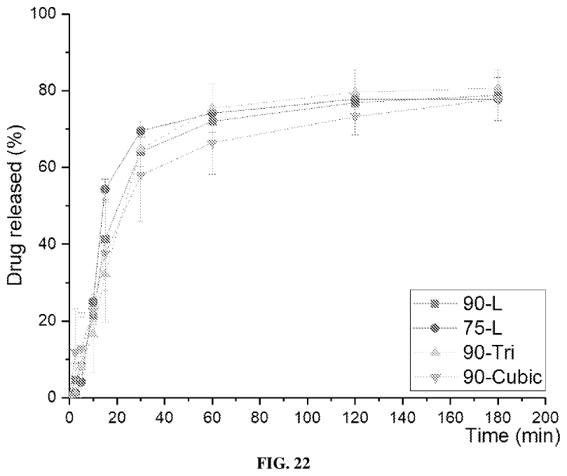

As shown in FIG. 22, all tablets showed identical drug release profiles, which can reach around 80% of drug release at 3 hours dissolution point. All tablets were floating initially during dissolution studies, mainly due to the lower densities compared to the dissolution media. As expected, the T_{CC-75L} showed faster drug release rates because of its more porous structure, while the T_{CC-90Cubic} showed a slower release rate due to the condense infill and specific layer structures. In fact, the infill patterns showed limited influence on the drug release performance where the release curve of T_{CC-90L} and T_{CC-90Tri} were almost overlapped since they have similar structures in the vertical directions.

Additionally, compared with the physical mixture tablets described above, the cocrystals-loaded tablets showed improved drug release performance due to the presence of the cocrystals in the 3D printed tablets.

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the methods and in the steps or in the sequence of steps of the method described herein without departing from the concept, spirit, and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope, and concept of the disclosure as defined by the appended claims.

What is claimed is:

1. A method of preparing a pharmaceutical composition comprising:
(A) obtain a composition comprising a co-crystal, wherein the co-crystal is of an active pharmaceutical ingredient and a co-former;
(B) subjecting the composition to an additive manufacturing technique to obtain a pharmaceutical composition.

2. The method of claim 1, wherein the composition comprises at least 50% of the active pharmaceutical ingredient and the co-former as a cocrystal.

3. The method of claim 2, wherein the composition comprises at least 75% of the active pharmaceutical ingredient and the co-former as a cocrystal.

4. The method of claim 3, wherein the composition comprises at least 90% of the active pharmaceutical ingredient and the co-former as a cocrystal.

5. The method of claim 4, wherein the composition comprises at least 95% of the active pharmaceutical ingredient and the co-former as a cocrystal.

6. The method of claim 5, wherein the composition comprises at least 97% of the active pharmaceutical ingredient and the co-former as a cocrystal.

7. The method of claim 5, wherein the composition comprises at least 99% of the active pharmaceutical ingredient and the co-former as a cocrystal.

8. The method according to any one of claims 1-7, wherein the co-crystal comprises an active pharmaceutical ingredient and a co-former in a molar ratio from about 1:10 to about 10:1.

9. The method of claim 8, wherein the molar ratio is from about 2:1 to about 1:2.

10. The method of claim 9, wherein the molar ratio is about 2:1.

11. The method of claim 9, wherein the molar ratio is about 1:1.

12. The method of claim 9, wherein the molar ratio is about 1:2.

13. The method according to any one of claims 1-12, wherein the composition present as a filament, a powder, a granule, or a particle.

14. The method of claim 13, wherein the composition is present as a filament.

15. The method of claim 13, wherein the composition is present as a powder or a granule.

16. The method according to any one of claims 1-15, wherein the active pharmaceutical ingredient is a BCS Class II drug.

17. The method according to any one of claims 1-15, wherein the active pharmaceutical ingredient is a BCS Class IV drug.

18. The method according to any one of claims 1-17, wherein the active pharmaceutical ingredient is an active pharmaceutical ingredient with a melting point of less than 250° C.

19. The method of claim 10, wherein the melting point is less than 200° C.

20. The method according to any one of claims 1-19, wherein the active pharmaceutical ingredient is selected from anticancer agents, antifungal agents, psychiatric agents such as analgesics, consciousness level-altering agents such as anesthetic agents or hypnotics, nonsteroidal anti-inflammatory agents (NSAIDs), anthelmintics, antiacne agents, antianginal agents, antiarrhythmic agents, anti-asthma agents, antibacterial agents, anti-benign prostate hypertrophy agents, anticoagulants, antidepressants, antidiabetics, antiemetics, antiepileptics, antigout agents, antihypertensive agents, anti-inflammatory agents, antimalarials, antimigraine agents, antimuscarinic agents, antineoplastic agents, anti-obesity agents, antiosteoporosis agents, antiparkinsonian agents, antiproliferative agents, antiprotozoal agents, antithyroid agents, antitussive agent, anti-urinary incontinence agents, antiviral agents, anxiolytic agents, appetite suppressants, beta-blockers, cardiac inotropic agents, chemotherapeutic drugs, cognition enhancers, contraceptives, corticosteroids, Cox-2 inhibitors, diuretics, erectile dysfunction improvement agents, expectorants, gastrointestinal agents, histamine receptor antagonists, immunosuppressants, keratolytics, lipid regulating agents, leukotriene inhibitors, macrolides, muscle relaxants, neuroleptics, nutritional agents, opioid analgesics, protease inhibitors, and sedatives.

21. The method of claim 20, wherein the active pharmaceutical ingredient is a chemotherapeutic drug, an antibiotic, or a nonsteroidal anti-inflammatory agent.

22. The method of claim 21, wherein the active pharmaceutical ingredient is a chemotherapeutic drug.

23. The method of claim 21, wherein the active pharmaceutical ingredient is an antibiotic.

24. The method of claim 21, wherein the active pharmaceutical ingredient is a nonsteroidal anti-inflammatory agent.

25. The method according to any one of claims 1-24, wherein the co-former interacts with the active pharmaceutical ingredient through one or more non-covalent interactions.

26. The method of claim 26, wherein the non-covalent interactions are ionic interactions, hydrogen bonding, halogen bonding, van der Waals forces, 7-71 interactions, or hydrophobic effects.

27. The method according to any one of claims 1-26, wherein the co-former and the active pharmaceutical ingredient interact with two or more non-covalent interactions.

28. The method according to any one of claims 1-27, wherein the co-former is a compound which modifies the solubility of the active pharmaceutical ingredient.

29. The method of claim 28, wherein the co-former is a compound which is sparingly soluble and modifies the solubility of the active pharmaceutical ingredient.

30. The method of claim 28, wherein the co-former is a compound which is sensitive to the environment and modifies the solubility of the active pharmaceutical ingredient.

31. The method of claim 30, wherein the compound is sensitive to the pH of the environment.

32. The method of claim 30, wherein the compound is sensitive to the temperature of the environment.

33. The method according to any one of claims 1-32, wherein the co-former is a compound that has no therapeutic effect.

34. The method according to any one of claims 1-32, wherein the co-former is a second active pharmaceutical ingredient.

35. The method of claim 34, wherein the second active pharmaceutical ingredient is for the same disease or disorder as the first active pharmaceutical ingredient.

36. The method of claim 34, wherein the second active pharmaceutical ingredient is for a different disease or disorder as the first active pharmaceutical ingredient.

37. The method according to any one of claims 1-36, wherein the co-former comprises one or more functional groups selected from amine, amide, a nitrogen containing heterocycle, carbonyl, carboxyl, hydroxyl, phenol, sulfone, sulfine, sulfinyl, sulfonyl, mercapto, and methyl thio.

38. The method of claim 37, wherein the functional group is a NH₂, OH, C(O), C(O)OH, SH, or a nitrogen containing heterocycle.

39. The method of claim 38, wherein the functional group is a nitrogen containing heterocycle, NH₂, OH, or SH.

40. The method according to any one of claims 1-39, wherein the co-former is a flavoring compound.

41. The method of claim 40, wherein the flavoring compound is saccharin.

42. The method according to any one of claims 1-39, wherein the co-former is a carboxylic acid.

43. The method of claim 42, wherein the co-former is maleic acid.

44. The method according to any one of claims 1-39, wherein the co-former is a vitamin or a vitamin derivative.

45. The method of claim 44, wherein the co-former is nicotinamide.

46. The method according to any one of claims 1-45, wherein the pKₐ of the active pharmaceutical ingredient and the pKₐ of the co-former have a pKₐ difference of less than 3.

47. The method of claim 46, wherein the pKₐ difference is less than 2.

48. The method of claim 47, wherein the pKₐ difference is less than 1.

49. The method of claim 48, wherein the pKₐ difference is less than 0.5.

50. The method according to any one of claims 1-49, wherein the composition further comprises an excipient.

51. The method of claim 50, wherein the excipient is a pharmaceutically acceptable thermoplastic polymer.

52. The method of claim 51, wherein the active pharmaceutical ingredient or the co-former is not soluble in the pharmaceutically acceptable thermoplastic polymer.

53. The method of claim 52, wherein the active pharmaceutical ingredient and the co-former are not soluble in the pharmaceutically acceptable thermoplastic polymer.

54. The method according to any one of claims 1-53, wherein the co-crystal has been prepared using hot-melt extrusion.

55. The method according to any one of claims 1-53, wherein the co-crystal has been prepared using a solvent evaporation method.

56. The method according to any one of claims 1-54, wherein the additive manufacturing technique is vat photopolymerization, material jetting, binder jetting, powder-bed fusion, material extrusion, directed energy deposition, or sheet lamination.

57. The method of claim 56, wherein the additive manufacturing technique is fused deposition modeling, binder spraying, or selective laser sintering.

58. The method according to any one of claims 1-57, wherein the additive manufacturing technique comprises exposing the composition to an energy source to form a pattern.

59. The method of claim 58, wherein the pattern is prepared by passing the energy source over the composition with a print speed from about 0.1 mm/s to about 50,000 mm/s.

60. The method of claim 59, wherein the print speed is from about 0.5 mm/s to about 1,000 mm/s.

61. The method of claim 60, wherein the print speed is from about 1 mm/s to about 250 mm/s.

62. The method of claim 61, wherein the print speed is 1 mm/s, 10 mm/s, 25 mm/s, 50 mm/s, or 75 mm/s.

63. The method according to any one of claims 1-62, wherein the energy source has a hatch spacing from about 0.1 μm to about 250 μm.

64. The method of claim 63, wherein the hatch spacing is from about 10 μm to about 200 μm.

65. The method of claim 64, wherein the hatch spacing is from about 10 μm to about 150 μm.

66. The method of claim 65, wherein the hatch spacing is about 25 μm or 120 μm.

67. The method according to any one of claims 1-66, wherein the method comprises exposing the composition to a laser in a pattern.

68. The method according to any one of claims 1-67, wherein the method comprises depositing a layer of the composition onto a surface in a chamber.

69. The method of claim 68, wherein the layer has a layer thickness from about 1 μm to about 5 mm.

70. The method of claim 69, wherein the layer thickness is from about 10 μm to about 2.5 mm.

71. The method of claim 70, wherein the layer thickness is from about 50 μm to about 1 mm.

72. The method of claim 71, wherein the layer thickness is from 50 μm to about 400 μm.

73. The method according to any one of claims 68-72, wherein the layer comprises a surface temperature at its surface different from a chamber temperature in the chamber.

74. The method of claim 73, wherein the surface temperature is from about 0° C. to about 250° C.

75. The method of claim 74, wherein the surface temperature is from about 50° C. to about 175° C.

76. The method of claim 75, wherein the surface temperature is from about 75° C. to about 150° C.

77. The method according to any one of claims 73-76, wherein the chamber temperature is from about 25° C. to about 250° C.

78. The method according to any one of claims 73-77, wherein the chamber temperature is from about 50° C. to about 200° C.

79. The method according to any one of claims 73-78, wherein the chamber temperature is from about 75° C. to about 150° C.

80. The method according to any one of claims 73-79, wherein the surface temperature is more than 5° C. less than the melting point of the co-crystal.

81. The method of claim 80, wherein the surface temperature is more than 10° C. less than the melting point of the composition.

82. The method according to any one of claims 67-81, wherein the energy source is a laser.

83. The method of claim 82, wherein the laser comprises a laser power from about 0.1 W to about 250 W.

84. The method of claim 83, wherein the laser power is from about 5 mW to about 20 W.

85. The method of claim 84, wherein the laser power is from about 50 mW to about 1 W.

86. The method of claim 85, wherein the laser power is from about 100 mW to about 500 mW.

87. The method according to any one of claims 82-86, wherein the laser comprises a beam size from about 0.25 μm to about 1 mm.

88. The method of claim 87, wherein the beam size is from about 1 μm to about 500 μm.

89. The method of claim 88, wherein the beam size is from about 2.5 μm to about 100 μm.

90. The method according to any one of claims 82-89, wherein the laser has a wavelength from about 50 nm to about 15,000 nm.

91. The method of claim 90, wherein the wavelength is from about 5 nm to about 11,000 nm.

92. The method of claim 91, wherein the wavelength is from about 200 nm to about 1,000 nm.

93. The method according to any one of claims 82-92, wherein the laser gives the composition an amount of energy equal to an electron laser density from about 2.5 J/mm³ to about 500 J/mm³.

94. The method of claim 93, wherein the electron laser density is from about 5 J/mm³ to about 250 J/mm³.

95. The method of claim 94, wherein the electron laser density is from about 7.5 J/mm³ to about 50 J/mm³.

96. The method according to any one of claims 93-95, wherein the electron laser density is greater than 2.5 J/mm³.

97. The method according to any one of claims 93-96, wherein the electron laser density is greater than 5 J/mm³.

98. The method according to any one of claims 93-97, wherein the electron laser density is greater than 7.5 J/mm³.

99. The method according to any one of claims 1-57, wherein the additive manufacturing technique comprises a method comprising:
(A) fusing the composition to obtain a fused composition; and
(B) extruding the fused composition through an extruder to obtain an extruded composition;
wherein the extruded composition is used to build an object and the object comprises a pattern.

100. The method of claim 99, wherein the object is built in a layer by layer fashion.

101. The method of either claim 99 or claim 100, wherein the extruder comprises a feeding step motor.

102. The method of claim 101, wherein the feeding step motor comprises a feeding gear, a hot end, and a nozzle.

103. The method according to any one of claims 1-57 and 99-102, wherein the pattern is prepared by passing the extruder over the composition.

104. The method of claim 103, wherein the extruder is passed over the composition with a print speed from about 0.1 mm/s to about 50,000 mm/s.

105. The method of claim 104, wherein the print speed is from about 0.5 mm/s to about 1,000 mm/s.

106. The method of claim 105, wherein the print speed is from about 1 mm/s to about 250 mm/s.

107. The method of claim 106, wherein the print speed is 1 mm/s, 10 mm/s, 25 mm/s, 50 mm/s, or 75 mm/s.

108. The method according to any one of claims 1-57 and 99-107, wherein the pattern is printed with a hatch spacing from about 0.1 μm to about 1 mm.

109. The method of claim 108, wherein the hatch spacing is from about 1 μm to about 500 μm.

110. The method of claim 109, wherein the hatch spacing is from about 10 μm to about 250 μm.

111. The method of claim 110, wherein the hatch spacing is about 25 μm or 120 μm.

112. The method according to any one of claims 1-57 and 99-107, wherein the object is built upon a building platform.

113. The method of claim 112, wherein the building platform is moved along in a Z-axis.

114. The method of either claim 112 or claim 113, wherein the building platform is heated to a first platform temperature from about 0° C. to about 250° C.

115. The method of claim 114, wherein the first platform temperature is from about 50° C. to about 175° C.

116. The method of claim 115, wherein the first platform temperature is from about 75° C. to about 150° C.

117. The method of either claim 112 or claim 113, wherein the building platform is heated to a second platform temperature from about −125° C. to about 25° C.

118. The method of claim 117, wherein the second platform temperature is from about −100° C. to about 0° C.

119. The method of claim 118, wherein the second platform temperature is from about −50° C. to about 0° C.

120. The method according to any one of claims 117-119, wherein the second platform temperature is sufficient to cool or solidify the object.

121. The method according to any one of claims 1-57 and 99-120, wherein the extruded composition is a filament.

122. The method of claim 121, wherein the method comprises using the filament in a fusion deposition modeling to obtain the object.

123. The method of claim 122, wherein the filament has a diameter from about 0.5 mm to about 10 mm.

124. The method of claim 123, wherein the diameter is from about 1 mm to about 7.5 mm.

125. The method of claim 124, wherein the diameter is from about 1.5 mm to about 5 mm.

126. The method of claim 125, wherein the diameter is either 1.75 mm or 3 mm.

127. The method according to any one of claims 121-126, wherein the filament has a strength such that the force needed to break the filament is greater than 1000 g.

128. The method of claim 127, wherein the strength of the filament is greater than 2000 g.

129. The method of claim 128, wherein the strength of the filament is greater than 3000 g.

130. The method according to any one of claims 121-129, wherein the filament has a strength such that the force needed to cut the filament is greater than 100 g.

131. The method of claim 130, wherein the strength of the filament is greater than 200 g.

132. The method of claim 131, wherein the strength of the filament is greater than 300 g.

133. The method according to any one of claims 121-132, wherein the filament has a stress such that the force needed to beak the filament is greater than 5,000 g/mm².

134. The method of claim 133, wherein the stress of the filament is greater than 10,000 g/mm².

135. The method of claim 134, wherein the stress of the filament is greater than 15,000 g/mm².

136. The method according to any one of claims 121-135, wherein the filament has a bend angle such that the force needed to break the filament is greater than 10°.

137. The method of claim 136, wherein the strength of the filament is greater than 20°.

138. The method of claim 137, wherein the strength of the filament is greater than 30°.

139. The method according to any one of claims 121-138, wherein the filament comprises an active pharmaceutical ingredient and an excipient.

140. The method of claim 139, wherein the filament comprises from about 10% w/w to about to 99% w/w of the active pharmaceutical ingredient.

141. The method according to any one of claims 1-57, wherein the additive manufacturing technique comprises a method comprising:
(A) depositing the composition to form a powder; and
(B) depositing a liquid binding material onto the powder.

142. The method of claim 141, wherein the method comprises repeating steps A and B.

143. The method of either claim 141 or claim 142, wherein steps A and B are repeated sufficient to create a pattern.

144. The method according to any one of claims 141-143, wherein the pattern is prepared by passing a binder jetting head over the composition with a print speed from about 0.1 mm/s to about 50,000 mm/s.

145. The method of claim 144, wherein the print speed is from about 0.5 mm/s to about 1,000 mm/s.

146. The method of claim 145, wherein the print speed is from about 1 mm/s to about 250 mm/s.

147. The method of claim 146, wherein the print speed is 1 mm/s, 10 mm/s, 25 mm/s, 50 mm/s, or 75 mm/s.

148. The method according to any one of claims 141-147, wherein the binder jetting head has a hatch spacing from about 50 μm to about 1 mm.

149. The method of claim 148, wherein the hatch spacing is from about 60 μm to about 500 μm.

150. The method of claim 149, wherein the hatch spacing is from about 80 μm to about 400 μm.

151. The method of claim 150, wherein the hatch spacing is about 100 μm.

152. The method according to any one of claims 141-151, wherein the liquid binding material has a viscosity from 0.1 mPa*s to 230 mPa*s.

153. The method of claim 152, wherein the viscosity is from about 0.25 mPa*s to 150 mPa*s.

154. The method of claim 153, wherein the viscosity is from about 0.5 mPa*s to 50 mPa*s.

155. The method according to any one of claims 141-154, wherein the powder has a particle size D₅₀ of the powder is less than about 50 μm.

156. The method according to any one of claims 141-154, wherein the powder has a particle size D₉₀ of the powder is greater than 50 μm.

157. The method of claim 156, wherein the particle size D₉₀ of the powder is less than 100 μm.

158. The method of claim 157, wherein the particle size D₉₀ of the powder is less than 80 μm.

159. The method according to any one of claims 141-158, wherein the powder has a Carr's Index of less than 25%.

160. The method of claim 159, wherein the Carr's Index is from about 15% to about 25%.

161. The method according to any one of claims 141-160, wherein the angle of repose of the powder is less than 40°.

162. The method of claim 161, wherein the angle of repose is from about 10° than 40°.

163. The method of claim 162, wherein the angle of repose is from about 15° than 30°.

164. The method of claim 163, wherein the angle of repose is from about 20° than 25°.

165. The method according to any one of claims 141-160, wherein the powder has a bulk density below 5 g/cm³.

166. The method of claim 165, wherein the bulk density is below 2.5 g/cm³.

167. The method of claim 166, wherein the bulk density is below 1.5 g/cm³.

168. The method according to any one of claims 1-167, wherein the pharmaceutical composition further comprises a filler.

169. The method of claim 168, wherein the filler is a salt.

170. The method of claim 169, wherein the salt is a calcium salt.

171. The method of claim 170, wherein the calcium salt is calcium sulfate.

172. The method according to any one of claims 168-171, wherein the pharmaceutical composition comprises from about 1% w/w to about 99% w/w of filler.

173. The method of claim 172, wherein the pharmaceutical composition comprises from about 25% w/w to about 98% w/w of filler.

174. The method of claim 173, wherein the pharmaceutical composition comprises from about 50% w/w to about 98% w/w of filler.

175. The method of claim 174, wherein the pharmaceutical composition comprises from about 75% w/w to about 97% w/w of filler.

176. The method according to any one of claims 1-175, wherein the pharmaceutical composition is deposited into a unit dose form.

177. The method of claim 176, wherein the unit dose form comprises two or more of distinct domains.

178. The method of claim 177, wherein each domain comprises a circular shape.

179. The method of either claim 177 or claim 178, wherein each domain comprises a height, a porosity, and either a core diameter for the central domain or an inner and outer diameter for domains around the core domain.

180. The method of claim 179, wherein the height is from 0.1 mm to about 50 mm.

181. The method of claim 180, wherein the height is from about 1 mm to about 25 mm.

182. The method of claim 181, wherein the height is from about 2.5 mm to about 10 mm.

183. The method according to any one of claims 179-182, wherein the porosity is from about 10% to about 100%.

184. The method of claim 183, wherein the porosity is from about 20% to about 90%.

185. The method of claim 184, wherein the porosity is from about 30% to about 80%.

186. The method of claim 183, wherein the porosity is from about 60% to about 100%.

187. The method of claim 186, wherein the porosity is from about 70% to about 100%.

188. The method according to any one of claims 179-187, wherein the core diameter is from about 0.1 mm to about 25 mm.

189. The method of claim 188, wherein the core diameter is from about 0.5 mm to about 10 mm.

190. The method of claim 189, wherein the core diameter is from about 1 mm to about 10 mm.

191. The method according to any one of claims 179-190, wherein the core diameter is equal to the inner diameter of the second domain.

192. The method of claim 191, wherein the inner diameter of the next domain is equal to the outer diameter of the preceding domain.

193. The method according to any one of claims 179-192, wherein the inner diameter is from about 0.1 mm to about 50 mm.

194. The method of claim 193, wherein the inner diameter is from about 0.5 mm to about 20 mm.

195. The method of claim 194, wherein the inner diameter is from about 1 mm to about 20 mm.

196. The method according to any one of claims 179-195, wherein the outer diameter is from about 0.2 mm to about 100 mm.

197. The method of claim 196, wherein the outer diameter is from about 1 mm to about 40 mm.

198. The method of claim 197, wherein the outer diameter is from about 2 mm to about 40 mm.

199. The method according to any one of claims 179-198, wherein the unit dose form comprises 2, 3, 4, or 5 domains.

200. The method of claim 199, wherein each domain has a different shape, porosity, height, or diameter.

201. The method according to any one of claims 1-98, wherein the pharmaceutical composition is a dosage form.

202. The method according to any one of claims 1-201, wherein the method further comprises milling the pharmaceutical composition into a dosage form.

203. The method according to any one of claims 1-202, wherein the dosage form is formulated for oral, pulmonary, nasal, topical, transdermal, or parenteral delivery.

204. The method according to any one of claims 1-203, wherein the dosage form is formulated for oral delivery.

205. The method of claim 204, wherein the oral delivery is formulated as a tablet, capsule, or suspension.

206. The method according to any one of claims 1-203, wherein the dosage form is formulated for topical delivery.

207. The method of claim 206, wherein the topical delivery is an emulsion, ointment, or cream.

208. The method according to any one of claims 1-203, wherein the dosage form is formulated for parenteral delivery.

209. The method of claim 208, wherein the parenteral delivery is a suspension, microemulsion, or depot.

210. A pharmaceutical composition prepared according to the methods according to any one of claims 1-209.

211. A method of treating or preventing a disease or disorder in a patient comprising administering to the patient in need thereof a therapeutically effective amount of a composition prepared according to the methods of any one of claims 1-210; wherein the active pharmaceutical ingredient is sufficient to treat or prevent the disease or disorder.
